(19) United States
(12) Patent Application Publication
(10) Pub. No.: US 2023/0094152 A1
(43) Pub. Date: Mar. 30, 2023

(54) VIRAL DELIVERY OF GAS VESICLE GENES

(71) Applicant: California Institute of Technology, Pasadena, CA (US)

(72) Inventors: Shirin Shivaei, Pasadena, CA (US); Mikhail G. Shapiro, Pasadena, CA (US)

(21) Appl. No.: 17/936,286

(22) Filed: Sep. 28, 2022

Related U.S. Application Data
(60) Provisional application No. 63/249,992, filed on Sep. 29, 2021.

(51) Int. Cl. C12N 15/86; C12N 15/63; A61B 8/08

(52) U.S. Cl. CPC C12N 15/86; C12N 15/635; A61B 8/08; C12N 2740/15043; C12N 2740/15071; C12N 2740/15045; C12N 2840/002

(57) ABSTRACT

Disclosed herein include methods, compositions, and kits suitable for use in imaging of in situ gene expression. There are provided, in some embodiments, viral vector compositions. Disclosed herein includes a single viral vector comprising one or more gas vesicle assembly (GVA) gene(s) encoding one or more GVA protein(s), and one or more gas vesicle structural (GVS) gene(s) encoding one or more GVS protein(s). The one or more GVA protein(s) and the one or more GVS protein(s) can be capable of forming gas vesicles (GVs) upon expression in a cell.

### RELATED APPLICATIONS

This application claims the benefit under 35 U.S.C. § 119(e) of U.S. Provisional Patent Application Ser. No. 63/249,992, filed Sep. 29, 2021, the content of this related application is incorporated herein by reference in its entirety for all purposes.

### STATEMENT REGARDING FEDERALLY SPONSORED R&D

This invention was made with government support under Grant Nos. NS120828 & EB018975 awarded by the National Institutes of Health. The government has certain rights in the invention.

### BACKGROUND

### Field

The present disclosure relates generally to the field of relates generally to the field of polynucleotide expression.

### Description of the Related Art

The discovery of the green fluorescent protein provided the ability to observe previously invisible biological processes that occur inside a cell. However, due to strong scattering of light, fluorescence imaging has limited penetration depth of about one millimeter inside intact tissue. As a result, studying cellular phenomena in mammalian organisms has been limited to cell culture, transparent animals or surgically accessible regions. An alternative imaging modality, ultrasound, has the ability to penetrate up to 10 cm inside the body with a high spatiotemporal resolution. Recent work has established gas vesicles (GVs), microbially derived gas-filled protein nanostructures, as genetically encodable acoustic reporter genes. Through buckling of their exterior protein shell, GVs can produce strong nonlinear acoustic contrast that sets them apart from linear scatterers in their surrounding environment. However, the use of GVs in mammalian cells has been so far limited to transfection-compatible cell lines and the need for tedious clonal expansions of sorted cells, due to the large gene cluster size of GVs (8-14 genes). To facilitate the use of acoustic reporter genes in a broader range of biological contexts such as primary cells and endogenous tissues, there is a need for an efficient, accessible, and a versatile platform to deliver these genes to cells both in vitro and in vivo.

### SUMMARY

Disclosed herein include viral vector compositions. In some embodiments, the viral vector composition comprises: a single viral vector comprising one or more first promoters operably connected to one or more gas vesicle (GV) polynucleotides comprising: one or more gas vesicle assembly (GVA) gene(s) encoding one or more GVA protein(s), and one or more gas vesicle structural (GVS) gene(s) encoding one or more GVS protein(s), wherein the one or more GVA protein(s) and the one or more GVS protein(s) are capable of forming gas vesicles (GVs) upon expression in a cell.

Disclosed herein include viral vector compositions. In some embodiments, the viral vector composition comprises: (i) a first viral vector comprising one or more first promoters operably connected to one or more gas vesicle (GV) polynucleotides comprising: one or more gas vesicle structural (GVS) gene(s) encoding one or more GVS protein(s); and (ii) a second viral vector comprising one or more first promoters operably connected to one or more GV polynucleotides comprising: one or more gas vesicle assembly (GVA) gene(s) encoding one or more GVA protein(s), wherein the one or more GVA protein(s) and the one or more GVS protein(s) are capable of forming gas vesicles (GVs) upon expression in a cell. In some embodiments, the first viral vector comprises one or more first promoters operably connected to one or more gas vesicle (GV) polynucleotides comprising: one or more gas vesicle assembly (GVA) gene(s) encoding one or more GVA protein(s).

Disclosed herein include viral vector compositions. In some embodiments, the viral vector composition comprises: (i) a primary viral vector comprising one or more first promoters operably connected to one or more gas vesicle (GV) polynucleotides comprising: one or more gas vesicle structural (GVS) gene(s) encoding one or more GVS protein(s); (ii) a secondary viral vector comprising one or more first promoters operably connected to one or more GV polynucleotides comprising: one or more gas vesicle assembly (GVA) gene(s) encoding one or more GVA protein(s); and (iii) a tertiary viral vector comprising one or more first promoters operably connected to one or more GV polynucleotides comprising: one or more gas vesicle assembly (GVA) gene(s) encoding one or more GVA protein(s), wherein the one or more GVA protein(s) and the one or more GVS protein(s) are capable of forming gas vesicles (GVs) upon expression in a cell. In some embodiments, the primary viral vector comprises one or more first promoters operably connected to one or more gas vesicle (GV) polynucleotides comprising: one or more gas vesicle assembly (GVA) gene(s) encoding one or more GVA protein(s).

In some embodiments, a viral vector comprises a polynucleotide encoding a detectable protein. In some embodiments, the detectable protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, mScarlet, derivatives thereof, or any combination thereof.

In some embodiments, a viral vector comprises: a context-dependent promoter operably linked to a transactivator polynucleotide comprising a transactivator gene, wherein the context-dependent promoter is capable of inducing transcription of the transactivator gene to generate a transactivator transcript, wherein the transactivator transcript is capable of being translated to generate a transactivator, wherein the activity of the context-dependent promoter and/or the degree of expression of the transactivator is associated with the presence and/or amount a unique cell type and/or a unique cell state, wherein, in the presence of the transactivator and a transactivator-binding compound, the first promoter is capable of inducing transcription of the one or more GV polynucleotides to generate GV transcript(s), and wherein the GV transcript(s) are capable of being translated to generate GVA protein(s) and/or GVS protein(s).

In some embodiments, the first promoter comprises one or more copies of a transactivator recognition sequence the transactivator is capable of binding to induce transcription, and wherein the transactivator is incapable of binding the transactivator recognition sequence in the absence of the transactivator-binding compound. In some embodiments, the one or more copies of a transactivator recognition sequence comprise one or more copies of a tet operator (TetO). In some embodiments, the first promoter comprises a tetracycline response element (TRE), and wherein the TRE comprises one or more copies of a tet operator (TetO). In some embodiments, the transactivator comprises reverse tetracycline-controlled transactivator (rtTA). In some embodiments, the transactivator comprises tetracycline-controlled transactivator (tTA). In some embodiments, the transactivator-binding compound comprises tetracycline, doxycycline or a derivative thereof. In some embodiments, the transactivator comprises a constitutive signal peptide for protein degradation (e.g., PEST).

In some embodiments, the degree of expression of the transactivator is positively correlated with the presence and/or amount a unique cell type and/or a unique cell state In some embodiments, a unique cell type and/or a unique cell state comprises a unique gene expression pattern. In some embodiments, the unique cell type and/or unique cell state comprises a unique anatomic location. In some embodiments, the unique cell type and/or the unique cell state comprises anatomically locally unique gene expression. In some embodiments, the context-dependent promoter is an Immediate Early Gene (IEG) regulated promoter (e.g., cFos, pRAM, or EGR1). In some embodiments, the unique cell state comprises activation of one or more cellular activities of interest, such as, for example, cellular activities associated with neural activity (e.g., fear conditioning, memory formation, learning, and/or sensory modalities). In some embodiments, a unique cell type and/or a unique cell state is caused by hereditable, environmental, and/or idiopathic factors. In some embodiments, a unique cell type and/or a unique cell state is caused by and/or associated with the expression of one or more endogenous proteins whose expression is regulated by the endogenous context-dependent promoter. In some embodiments, the unique cell state and/or unique cell type is characterized by signaling of one or more endogenous signal transducer(s) (e.g., signal transducer(s) regulated by the endogenous context-dependent promoter).

In some embodiments, the unique cell type and/or the cell in the unique cell state (i) causes and/or aggravates a disease or disorder and/or (ii) is associated with the pathology of a disease or disorder. In some embodiments, the unique cell state comprises: a physiological state (e.g., a cell cycle state, a differentiation state, a development state, a metabolic state, or a combination thereof); and/or a pathological state (e.g., a disease state, a human disease state, a diabetic state, an immune disorder state, a neurodegenerative disorder state, an oncogenic state, or a combination thereof). In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

In some embodiments, the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the context-dependent promoter comprises a tissue-specific promoter and/or a lineage-specific promoter. In some embodiments, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a hSynapsin promoter, a α-myosin heavy chain (a-MHC) promoter, or a cardiac Troponin T (cTnT) promoter. In some embodiments, the tissue specific promoter is a neuronal activity-dependent promoter and/or a neuron-specific promoter (e.g., a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ 1.7 promoter, a Naᵥ 1.8 promoter, a Naᵥ 1.9 promoter, or an Advillin promoter). In some embodiments, the tissue specific promoter is a muscle-specific promoter. In some embodiments, the muscle-specific promoter comprises a creatine kinase (MCK) promoter.

In some embodiments, the GVA genes and/or GVS genes are derived from Bacillus megaterium, Anabaena flos-aquae, Serratia sp., Bukholderia thailandensis, B. megaterium, Frankia sp, Haloferax mediaterranei, Halobacterium sp, Halorubrum vacuolatum, Microcystis aeruginosa, Methanosarcina barkeri, Streptomyces coelicolor, and/or Psychromonas ingrahamii. In some embodiments, the one or more GV polynucleotides comprise: two or more GVS genes derived from different prokaryotic species; GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii; gvpB, gvpN gvpF, gvpG, gvpL gvpS, gvpK, gvpJ, and/or gvpU from B. megaterium; gvpA, gvpC, gvpN, gvpJ, gvpK, gvpF, gvpG, gvpV, and/or gvpW from Anabaena flos-aquae; gvpR, gvpN, gvpF, gvpG, gvpL, gvpS, gvpK, gvpJ, gvpT and/or gvpU from B. megaterium and gvpA from Anabaena flos-aquae; gvpA, and/or gvpC from Anabaena flos-aquae, and gvpN, gvpF, gvpG, gvpL, gvpS, gvpK, gvpJ, and/or gvpU from B. megaterium; and/or gvpA, gvpC and/or gvpN from Anabaena flos-aquae, and gvpF, gvpG, gvpL, gvpS, gvpK, gvpJ, and/or gvpU from B. megaterium. In some embodiments, the GVA genes and GVS genes have sequences codon optimized for expression in a eukaryotic cell. In some embodiments, the GVs comprise a GVS variant engineered to present a tag enabling clustering in the cell. In some embodiments, the GVs are hybrid GVs derived from two or more prokaryotic species.

In some embodiments, the single viral vector comprises: a first GV polynucleotide encoding GvpA, a second GV polynucleotide encoding GvpN, a third GV polynucleotide encoding GvpJ, a fourth GV polynucleotide encoding GvpK, a fifth GV polynucleotide encoding GvpF, a sixth GV polynucleotide encoding GvpG, a seventh GV polynucleotide encoding GvpW, and an eighth GV polynucleotide encoding GvpV. In some embodiments, each of the GV polynucleotides is operably connected to a tandem gene expression element.

In some embodiments, the first viral vector comprises: a first GV polynucleotide encoding GvpA, and wherein the second viral vector comprises: a second GV polynucleotide encoding GvpN, a third GV polynucleotide encoding GvpJ, a fourth GV polynucleotide encoding GvpK, a fifth GV polynucleotide encoding GvpF, a sixth GV polynucleotide encoding GvpG, a seventh GV polynucleotide encoding GvpW, and an eighth GV polynucleotide encoding GvpV. In some embodiments, each of the GV polynucleotides is operably connected to a tandem gene expression element.

In some embodiments, the primary viral vector comprises: a first GV polynucleotide encoding GvpA, wherein the secondary viral vector comprises: a second GV polynucleotide encoding GvpG, a third GV polynucleotide encoding GvpW, and a fourth GV polynucleotide encoding GvpV, and wherein the tertiary viral vector comprises: a fifth GV polynucleotide encoding GvpN, a sixth GV polynucleotide encoding GvpJ, a seventh GV polynucleotide encoding GvpK, and an eighth GV polynucleotide encoding GvpF. In some embodiments, each of the GV polynucleotides is operably connected to a tandem gene expression element.

In some embodiments, the plurality of gas vesicles comprises a first collapse pressure profile. In some embodiments, the first collapse pressure profile comprises a collapse function from which a gas vesicle collapse amount can be determined for a given pressure value. In some embodiments, the first collapse pressure profile comprises a first initial collapse pressure where 5% or lower of the plurality of gas vesicles are collapsed, a first midpoint collapse pressure where 50% of the plurality of gas vesicles are collapsed, a first complete collapse pressure where at least 95% of the plurality of gas vesicles are collapsed, any pressure between the first initial collapse pressure and the first midpoint collapse pressure, and any pressure between the first midpoint collapse pressure and the first complete collapse pressure. In some embodiments, a first selectable collapse pressure is: any collapse pressure within the first collapse pressure profile; selected from the first collapse pressure profile at a value between 0.05% collapse of the plurality of gas vesicles and 95% collapse of the plurality of gas vesicles; equal to or greater than the first initial collapse pressure; equal to or greater than the first midpoint collapse pressure; and/or equal to or greater than the first complete collapse pressure.

In some embodiments, the plurality of gas vesicles comprises a first buckling pressure profile. In some embodiments, the first collapse pressure profile comprises a buckling function from which a gas vesicle buckling amount can be determined for a given pressure value. In some embodiments, the buckling amount comprises the amount of nonlinear contrast. In some embodiments, the first buckling pressure profile comprises a first buckling threshold pressure where the gas vesicles starts to buckle and produce nonlinear contrast, a first optimum buckling pressure where the gas vesicles exhibit maximum buckling and produce the highest level of nonlinear contrast, a first collapse pressure wherein the gas vesicles collapse, any pressure between the first buckling threshold pressure and the first optimum buckling pressure, and any pressure between the first optimum buckling pressure and the first collapse pressure. In some embodiments, a first selectable buckling pressure is: any collapse pressure within the first buckling pressure profile; the first optimum buckling pressure; and/or equal to or less than the first initial collapse pressure.

In some embodiments, the viral vector is or comprises an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector derivatives thereof, or any combination thereof, In some embodiments, the viral vector is capable of integrating into a mammalian cell genome, and wherein the GVs are capable of being expressed upon integration.

In some embodiments, the viral vector is a lentiviral vector (e.g., human immunodeficiency virus 1 (HIV-1), human immunodeficiency virus 2 (HIV-2), visna-maedi virus (VMV) virus, caprine arthritis-encephalitis virus (CAEV), equine infectious anemia virus (EIAV), feline immunodeficiency virus (FIV), bovine immune deficiency virus (BIV), simian immunodeficiency virus (SIV), derivatives thereof, or any combination thereof). In some embodiments, the viral vector is a recombinant lentiviral vector. In some embodiments, the recombinant lentiviral vector is derived from a lentivirus pseudotyped with vesicular stomatitis virus (VSV), lymphocytic choriomeningitis virus (LCMV), Ross river virus (RRV), Ebola virus, Marburg virus, Mokala virus, Rabies virus, RD114, or variants therein.

The viral vector composition (e.g., a single viral vector, a first viral vector, a second viral vector, a primary viral vector, a secondary viral vector, a tertiary viral vector) can comprise: one or more of a left (5′) retroviral LTR, a Psi (Ψ) packaging signal, a central polypurine tract/DNA flap (cPPT/FLAP), a retroviral export element, and a right (3′) retroviral LTR. In some embodiments, the promoter of the 5′ LTR is replaced with a heterologous promoter. In some embodiments, the 5′ LTR or 3′ LTR is a lentivirus LTR. In some embodiments, the 3′ LTR comprises one or more modifications and/or deletions. In some embodiments, the 3′ LTR is a self-inactivating (SIN) LTR.

In some embodiments, the AAV vector comprises single-stranded AAV (ssAAV) vector or a self-complementary AAV (scAAV) vector. In some embodiments, the AAV vector comprises AAV1, AAV2, AAV3, AAV4, AAV5, AAV6, AAV7, AAV8, AAV9, derivatives thereof, or any combination thereof. In some embodiments, the AAV vector comprises an AAV9 variant engineered for systemic delivery (e.g., AAV-PHP.B, AAV-PHP.eB, or AAV-PHP.S).

In some embodiments, one or more GV polynucleotides and/or transactivator polynucleotide comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the one or more first promoters comprise: a minimal promoter (e.g., TATA, miniCMV, and/or miniPromo); a tissue-specific promoter and/or a lineage-specific promoter; and/or a ubiquitous promoter (e.g., a minEf1a promoter, a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof). In some embodiments, the viral vector is encapsidated in a viral particle.

Disclosed herein include methods of imaging a target site of a subject. In some embodiments, the method comprises: administering to the subject an effective amount of a viral vector composition disclosed herein; and applying ultrasound (US) to the target site of the subject to obtain a US image of the target site, optionally a nonlinear US image.

Disclosed herein include methods of imaging gene expression within a subject. In some embodiments, the method comprises: administering to the subject an effective amount of a viral vector composition disclosed herein; and applying ultrasound (US) to the target site of the subject to obtain a US image of gene expression at the target site, optionally a nonlinear US image.

Disclosed herein include methods of imaging gene expression within target cells. In some embodiments, the method comprises: transducing target cells with a viral vector composition disclosed herein; and applying ultrasound (US) to said target cells to obtain a US image of gene expression at the target site, optionally a nonlinear US image. The target cells can be target cells of a subject. The transducing step can comprise administering to the subject an effective amount of a viral vector composition disclosed herein.

Disclosed herein include methods of detecting a unique cell type and/or unique cell state within a subject. In some embodiments, the method comprises: administering to the subject an effective amount of a viral vector composition disclosed herein; and applying ultrasound (US) to the target site of the subject, thereby detecting the unique cell type and/or unique cell state within said subject.

Disclosed herein include methods of detecting a unique cell type and/or unique cell state. In some embodiments, the method comprises: transducing target cells with a viral vector composition disclosed herein; and applying ultrasound (US) to said target cells, thereby detecting the unique cell type and/or unique cell state. The target cells can be target cells of a subject. The transducing step can comprise administering to the subject an effective amount of a viral vector composition disclosed herein.

Disclosed herein include methods of detecting perturbation-induced changes in cell type and/or cell state. In some embodiments, the method comprises: transducing target cells with a viral vector composition disclosed herein; introducing one or more genetic, chemical, and/or physical perturbations to said target cells; and applying ultrasound (US) to said target cells, thereby detecting the perturbation-induced changes in cell type and/or cell state. The target cells can be target cells of a subject. The transducing step can comprise administering to the subject an effective amount of a viral vector composition disclosed herein.

In some embodiments, the administering step comprises: isolating target cells from the subject; transducing target cells with the viral vector composition disclosed herein; and administering to the subject an effective amount of the transduced cells. The method can comprise: administering an effective amount of a transactivator-binding compound to the subject. In some embodiments, the transactivator-binding compound comprises tetracycline, doxycycline or a derivative thereof. In some embodiments, the administering comprises systemic administration. In some embodiments, the systemic administration is intravenous, intramuscular, intraperitoneal, or intraarticular. In some embodiments, administering comprises intracranial delivery, intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, the period of time between the administering and applying is about 21 days, about 14 days, about 7 days, about 3 days, about 48 hours, about 44 hours, about 40 hours, about 35 hours, about 30 hours, about 25 hours, 20 hours, 15 hours, 10 hours, about 8 hours, about 7 hours, about 6 hours, about 5 hours, about 4 hours, about 3 hours, about 2 hours, about 1 hour, about 30 minutes, about 15 minutes, about 10 minutes, or about 5 minutes.

In some embodiments, the target cell(s) comprises an immortalized cells or primary cells. In some embodiments, the target cell comprises a eukaryotic cell. In some embodiments, the eukaryotic cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the target cells are situated within a target site of a subject, and wherein applying US comprises applying US to the target site to obtain a US image of the target site. In some embodiments, the target cells in a unique cell type and/or a unique cell state express GVs, and wherein said GVs produce nonlinear ultrasound contrast. In some embodiments, applying US causes the production of unique cell type-dependent and/or unique cell state-dependent nonlinear ultrasound contrast. In some embodiments, detecting a unique cell type and/or unique cell state comprises detecting an at least about 5 dB enhancement in nonlinear ultrasound contrast. In some embodiments, applying US comprises nonlinear US imaging. In some embodiments, applying US comprises applying one or more US pulse(s) over a duration of time. In some embodiments, the duration of time is about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes.

In some embodiments, the one or more US pulse(s) each have a pulse duration of about 1 hour, about 30 minutes, about 15 minutes, about 10 minutes, about 5 minutes, about 1 minute, about 1 second, or about 1 millisecond. In some embodiments, applying one or more US pulse(s) comprises applying one or more focused US pulse(s). In some embodiments, applying one or more US pulse(s) comprises applying US at a frequency of 100 kHz to 100 MHz. In some embodiments, applying one or more US pulse(s) comprises applying ultrasound at a frequency of 0.2 to 1.5 mHz. In some embodiments, applying one or more US pulse(s) comprises applying ultrasound having a mechanical index in a range between 0.2 and 0.6. In some embodiments, the one or more US pulse(s) comprise a peak pressure of about 40 kPa to about 800 kPa. In some embodiments, the one or more US pulse(s) comprise a peak pressure of about 70 kPa to about 150 kPa, and/or about 440 kPa to about 605 kPa. In some embodiments, the one or more US pulse(s) comprise a pressure value that is: the selectable buckling pressure; the first optimum buckling pressure; and/or equal to or less than the first initial collapse pressure.

In some embodiments, the one or more US pulse(s) induces collapse of GVs, and wherein the one or more US pulse(s) comprise a pressure value that is the first selectable collapse pressure. In some embodiments, the nonlinear ultrasound imaging comprises cross-amplitude modulation (x-AM) ultrasound imaging or parabolic amplitude modulation (pAM) ultrasound imaging. In some embodiments, the nonlinear ultrasound imaging comprises differential nonlinear ultrasound imaging. In some embodiments, differential nonlinear ultrasound imaging comprises imaging of the second and/or higher harmonics with the first harmonic signal subtracted out. In some embodiments, the nonlinear ultrasound imaging comprises cross-phase modulation imaging and/or harmonic imaging. In some embodiments, the nonlinear ultrasound imaging comprises providing amplitude modulation (AM) ultrasound pulse sequences.

In some embodiments, the nonlinear ultrasound imaging comprises: pairs of cross-propagating plane waves to elicit nonlinear scattering from buckling GVs at the wave intersection; subtracting the signal generated by transmitting each wave on its own; and quantifying the resulting contrast. In some embodiments, the signals generated by transmitting each wave on its own has linear characteristics and/or lower nonlinear characteristics than the combined transmission of both plane waves produced at their intersection. In some embodiments, the nonlinear ultrasound imaging comprises: a peak positive pressure of two single tilted plane waves exciting the GV in a linear scattering regime; a doubled X-wave intersection amplitude exciting the GV in a nonlinear scattering regime; summing the echoes from the two single tilted plane-wave transmissions to generate a sum; and subtracting the sum from the echoes of the X-wave transmissions to derive nonzero differential GV signals.

In some embodiments, applying US comprises detecting scattering of the one or more US pulse(s) by gas vesicles (e.g., nonlinear scattering of the US by buckling gas vesicles). In some embodiments, detecting scattering comprises: detecting backscattered echoes of two half-amplitude transmissions at applied pressures below the buckling threshold of the GV; and detecting backscattered echoes of a third full-amplitude transmission at pressures above the buckling threshold of the gas vesicles. In some embodiments, said two half-amplitude transmissions trigger largely linear scattering. In some embodiments, said third full-amplitude transmission triggers harmonic and nonlinear scattering. In some embodiments, the method comprises subtracting the backscattered echoes of the two half-amplitude transmissions from the backscattered echoes of the third full-amplitude transmission.

In some embodiments, the method comprises: single-cell imaging; and/or imaging a large volume in deep tissue. In some embodiments, the method comprises US imaging with a spatiotemporal resolution of less than about 100 μm and less than about 1 ms. In some embodiments, the target site comprises: a volume larger than about 1 mm³; a depth deeper than about 1 mm; a depth and/or a volume inaccessible via optical imaging and/or fiber photometry; and/or the entire brain or a portion thereof. In some embodiments, the target cells are in vitro, in vivo, and/or ex vivo. In some embodiments, the target cells are tissue culture cells. In some embodiments, the subject is a mammal. In some embodiments, the subject is not anesthetized. In some embodiments, the target site comprises a site of disease or disorder or is proximate to a site of a disease or disorder. In some embodiments, the target site comprises a tissue. In some embodiments, the tissue is inflamed tissue and/or infected tissue. In some embodiments, the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue comprises: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

In some embodiments, the nonlinear ultrasound imaging comprises transcranial imaging, wherein the target site comprises one or more target brain region(s), and wherein the target brain region(s) comprises the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kolliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (S1), Secondary somatosensory cortex (S2), Posterior parietal cortex, postcentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof. In some embodiments, the brain region comprises neural pathways Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

### BRIEF DESCRIPTION OF THE DRAWINGS

FIGS. 1A-1B depict non-limiting exemplary schematics related to the structure and acoustic properties of GVs. FIG. 1A depicts schematics showing gas-filled protein nanostructures with an inner hydrophobic shell that excludes air from inside the structure and a hydrophilic outer shell. FIG. 1B depicts schematics showing that below a certain acoustic pressure, GVs act as linear scatterers where the intensity of scattering linearly increases with pressure. Above their buckling pressure, GVs resonate at harmonic frequencies of the source, creating nonlinear contrast. High pressure eventually results in full collapse of GVs.

FIGS. 2A-2F depict non-limiting exemplary schematics and data related to GV-encoding viral architectures and transduction of HEK293T cells. FIG. 2A depicts combinations of lentiviral transgene vectors that together encode all necessary genes for GV formation, driven by the Tet-ON expression system. FIG. 2B depicts data related to HEK293T cells transduced with the dual lentiviral vector design (top) or the tri-vector design (bottom) and induced with doxycycline (1 mg/mL) for 72 hours. The nonlinear signal disappears in the same sample through collapse of GVs.

FIG. 2C depicts data related to nonlinear contrast in the transduced cells increasing with acoustic pressure due to enhanced buckling of GVs. n=2, technical replicates. Error bars indicate SEM and are only shown when larger than the symbols. FIGS. 2D-2F depict a non-limiting exemplary schematic (FIG. 2D) and data (FIGS. 2E-2F) related to a single lentiviral vector encoding all GV genes under the TRE promoter. HEK-TetON cells transduced with the virus and induced with doxycycline (1 mg/mL) for 72 hours show nonlinear contrast. n=2, technical replicates. Error bars indicate SEM and are only shown when larger than the symbols.

FIGS. 3A-3D depict data related to lentiviral delivery of GV genes to various cell types. FIGS. 3A-3B depict data related to nonlinear imaging (xAM) of U87, N2a, and Jurkat cells transduced with one of the two designed viral combinations, induced with doxycycline (1 mg/mL) for 72 hours. n=2, technical replicates. Error bars indicate SEM and are only shown when larger than the symbols. FIG. 3C depicts data related to nonlinear imaging (xAM) of cultured cortical neurons extracted from embryonic mouse brain, transduced with the tri-viral vector transgenes. Here the rtTA transactivatior was expressed under minEf1a or hSynapsin. No GVs control contains neurons transduced with an AAV-GCaMP expressing virus without any GV genes. FIG. 3D depicts data related to TEM grids of GVs floating in the lysate of cortical neurons transduced with the tri-virus system under hSynapsin promoter. Scale bar=100 nm.

FIGS. 4A-4B depict a non-limiting exemplary schematic and data related to activity inducible expression of GVs in HEK293T cells. FIG. 4A depicts a non-limiting exemplary schematic showing a pipeline for induction of activity in HEK293T transduced cells. Cells were induced with ionomycin at 1.3 μM and doxycycline at 1 mg/mL for 6 hours at which point the media was changed with tetracycline-free cell culture media. FIG. 4B depicts data related to nonlinear (xAM) contrast from HEK293T cells expressing GVs under activity-dependent promoters. Cells with activity induction received ionomycin (1.3 uM) and doxycycline (1 mg/mL) for 6 hours, and cells without activity induction received only doxycycline (1 mg/mL). n=2, technical replicates. Error bars indicate SEM and are only shown when larger than the symbols.

FIGS. 5A-5C depict a non-limiting exemplary schematic and data related to US imaging of in situ gene expression in the brain. FIG. 5A depicts a non-limiting exemplary schematic showing a pipeline for intracranial injection of GV-encoding lentiviruses and transcranial imaging of the transduced region. FIGS. 5B-5C depict data related to transcranial nonlinear imaging (parabolic AM) of the brain of mouse 1 (FIG. 5B) and mouse 2 (FIG. 5C) overlaid on functional ultrasound imaging (fUS). GV expression was induced for 24 hours prior to imaging via IP injection of doxycycline (75-100 ug per 20 kg body mass).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include viral vector compositions. In some embodiments, the viral vector composition comprises: a single viral vector comprising one or more first promoters operably connected to one or more gas vesicle (GV) polynucleotides comprising: one or more gas vesicle assembly (GVA) gene(s) encoding one or more GVA protein(s), and one or more gas vesicle structural (GVS) gene(s) encoding one or more GVS protein(s), wherein the one or more GVA protein(s) and the one or more GVS protein(s) are capable of forming gas vesicles (GVs) upon expression in a cell.

Disclosed herein include viral vector compositions. In some embodiments, the viral vector composition comprises: (i) a first viral vector comprising one or more first promoters operably connected to one or more gas vesicle (GV) polynucleotides comprising: one or more gas vesicle structural (GVS) gene(s) encoding one or more GVS protein(s); and (ii) a second viral vector comprising one or more first promoters operably connected to one or more GV polynucleotides comprising: one or more gas vesicle assembly (GVA) gene(s) encoding one or more GVA protein(s), wherein the one or more GVA protein(s) and the one or more GVS protein(s) are capable of forming gas vesicles (GVs) upon expression in a cell. In some embodiments, the first viral vector comprises one or more first promoters operably connected to one or more gas vesicle (GV) polynucleotides comprising: one or more gas vesicle assembly (GVA) gene(s) encoding one or more GVA protein(s).

Disclosed herein include viral vector compositions. In some embodiments, the viral vector composition comprises: (i) a primary viral vector comprising one or more first promoters operably connected to one or more gas vesicle (GV) polynucleotides comprising: one or more gas vesicle structural (GVS) gene(s) encoding one or more GVS protein(s); (ii) a secondary viral vector comprising one or more first promoters operably connected to one or more GV polynucleotides comprising: one or more gas vesicle assembly (GVA) gene(s) encoding one or more GVA protein(s); and (iii) a tertiary viral vector comprising one or more first promoters operably connected to one or more GV polynucleotides comprising: one or more gas vesicle assembly (GVA) gene(s) encoding one or more GVA protein(s), wherein the one or more GVA protein(s) and the one or more GVS protein(s) are capable of forming gas vesicles (GVs) upon expression in a cell. In some embodiments, the primary viral vector comprises one or more first promoters operably connected to one or more gas vesicle (GV) polynucleotides comprising: one or more gas vesicle assembly (GVA) gene(s) encoding one or more GVA protein(s).

Disclosed herein include methods of imaging a target site of a subject. In some embodiments, the method comprises: administering to the subject an effective amount of a viral vector composition disclosed herein; and applying ultrasound (US) to the target site of the subject to obtain a US image of the target site, optionally a nonlinear US image.

Disclosed herein include methods of imaging gene expression within a subject. In some embodiments, the method comprises: administering to the subject an effective amount of a viral vector composition disclosed herein; and applying ultrasound (US) to the target site of the subject to obtain a US image of gene expression at the target site, optionally a nonlinear US image.

Disclosed herein include methods of imaging gene expression within target cells. In some embodiments, the method comprises: transducing target cells with a viral vector composition disclosed herein; and applying ultrasound (US) to said target cells to obtain a US image of gene expression at the target site, optionally a nonlinear US image. The target cells can be target cells of a subject. The transducing step can comprise administering to the subject an effective amount of a viral vector composition disclosed herein.

Disclosed herein include methods of detecting a unique cell type and/or unique cell state within a subject. In some embodiments, the method comprises: administering to the subject an effective amount of a viral vector composition disclosed herein; and applying ultrasound (US) to the target site of the subject, thereby detecting the unique cell type and/or unique cell state within said subject.

Disclosed herein include methods of detecting a unique cell type and/or unique cell state. In some embodiments, the method comprises: transducing target cells with a viral vector composition disclosed herein; and applying ultrasound (US) to said target cells, thereby detecting the unique cell type and/or unique cell state. The target cells can be target cells of a subject. The transducing step can comprise administering to the subject an effective amount of a viral vector composition disclosed herein.

Disclosed herein include methods of detecting perturbation-induced changes in cell type and/or cell state. In some embodiments, the method comprises: transducing target cells with a viral vector composition disclosed herein; introducing one or more genetic, chemical, and/or physical perturbations to said target cells; and applying ultrasound (US) to said target cells, thereby detecting the perturbation-induced changes in cell type and/or cell state. The target cells can be target cells of a subject. The transducing step can comprise administering to the subject an effective amount of a viral vector composition disclosed herein.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

The term “brain cell” as used herein shall be given its ordinary meaning and shall also refer to cells that form the brain of an individual with the exclusion of blood vessels and meninges (dura mater, arachnoid mater, and pia mater in mammals) of the individual. Exemplary brain cells comprise neurons and glial cells.

The terms “neuron”, “nerve cell or “neural cell” as used herein interchangeably shall be given their ordinary meaning and also indicate an electrically excitable cell that receives, processes, and transmits information through electrical and chemical signals. A neuron consists of a cell body (or soma) which contains the neuron's nucleus (with DNA and typical nuclear organelles), branching dendrites (signal receivers) and a projection called axon, which take information away from the cell body and conduct the nerve signal. At the other end of the axon, axon terminals transmit the electro-chemical signal across a synapse (the gap between the axon terminal and the receiving cell). Accordingly, neural brain cells are nerve cells of the brain that transmit nerve signals to and from the brain.

Brain cells are comprised within areas of the brain defined as gray matter and white matter. The gray matter indicates an area of the brain comprising primarily neuronal cell bodies, neuropil (dendrites and myelinated as well as unmyelinated axons), glial cells (astrocytes and microglia), and synapses. White matter indicates an area of the brain which mainly comprise myelinated axons, also called tracts.

Brain cells are also comprised within “brain regions” which are areas anatomically defined by appearance and position as well as by their locations and their relationships with other parts of the brain. Exemplary brain regions can comprise the medulla (region containing many small nuclei involved in a wide variety of sensory and involuntary motor functions such as vomiting, heart rate and digestive processes), the pons (region of the brainstem directly above the medulla, which contains nuclei that control often voluntary but simple acts such as sleep, respiration, swallowing, bladder function, equilibrium, eye movement, facial expressions, and posture, includes) the hypothalamus (small region at the base of the forebrain composed of numerous small nuclei, each with distinct connections and neurochemistry, and engaged in additional involuntary or partially voluntary acts such as sleep and wake cycles, eating and drinking, and the release of some hormones), the thalamus (a region of nuclei with diverse functions such as relaying information to and from the cerebral hemispheres, motivation, and action-generating systems such as the action generating systems for several types of “consummatory” behaviors such as eating, drinking, defecation, and copulation, in the subthalamic area also zona incerta), the cerebellum (a region modulating the outputs of other brain regions, whether motor related or thought related, to make them certain and precise), the optic tectum (a region usually referred to as the superior colliculus in mammals, allowing actions such as eye movements and reaching movements to be directed toward points in space, most commonly in response to visual input), the pallium (a region of gray matter that lies on the surface of the forebrain also identified in reptiles and mammals as cerebral cortex which with multiple functions including smell and spatial memory), the hippocampus, (a region involved in complex events such as spatial memory and navigation in fishes, birds, reptiles, and mammals), the basal ganglia (a region involved in action selection as the related brain cells send inhibitory signals to all parts of the brain that can generate motor behaviors, and in the right circumstances release the inhibition, it comprises regions such as caudate nucleus, putamen, globus pallidus, substantia nigra, subthalamic nucleus, nucleus accumbens) and the olfactory bulb (a region that processes olfactory sensory signals and sends its output to the olfactory part of the pallium.

In some embodiments brain cells are further comprised in neural circuits possibly comprising cells and regions of additional parts of the body including cells of the peripheral nervous systems and other systems and organs of the body of the individual.

The wording “neural circuits” shall be given its ordinary meaning and shall also refer to a population of cells including neurons interconnected by synapses to pass an electrochemical signal from a neuron to another to carry out a specific function when activated. In some embodiments, the specific function neural circuits herein described manifests in a behavior or physiological function of the individual.

The term “behavior” as used herein shall be given its ordinary meaning and shall also refer to an internally coordinated responses (actions or inactions) of a whole living individual to internal and/or external stimuli. Exemplary behaviors in the sense of the disclosure comprise eating, drinking, defecation, and copulation, speaking, contemplating, remembering, focusing attention and additional behaviors identifiable by a skilled person.

The wording “physiological function” as used herein shall be given its ordinary meaning and shall also refer to a series of action and reactions performed by components of a living organism such as organ systems, organs, cells, and biomolecules to carry out the chemical and physical functions that exist in the living system. Exemplary physiological functions comprise action and reactions performed by components of the organism of an individual to carry out digestion of food, circulation of blood, contraction of muscles as well as other biophysical and biochemical phenomena, related to the coordinated homeostatic control mechanisms, and the continuous communication between cells in a living organism.

Neural circuits control behaviors and physiological function of an individual and changes in activity of neural circuits can lead to changes in behaviors and physiological functions of an individual as will be understood by a skilled person.

Exemplary neural circuit comprise the trisynaptic circuit in the hippocampus. the Papez circuit linking the hypothalamus to the limbic lobe, and neural circuits in the cortico-basal ganglia-thalamo-cortical loop which transmit information from the cortex, to basal ganglia, and thalamus, and back to the cortex, as well as the microcircuitry internal to the striatum the largest structure within the basal ganglia and additional circuits identifiable by a skilled person.

Methods and systems of the disclosure and related vectors and compositions in some embodiments only target brain cells whose cell bodies, dendrites or synapses are located in the gray matter. Accordingly in some embodiments “target brain cell” refers only to brain cells of the gray matter and the wording “target brain regions” only refer to brain regions comprising target brain cells, such as cerebral cortex, cerebellum, thalamus; hypothalamus; subthalamus, basal ganglia such as putamen, globus pallidus, nucleus accumbens; septal nuclei, deep cerebellar nuclei, dentate nucleus, globose nucleus, emboliform nucleus, fastigial nucleus), brainstem and regions thereof such as sub stantia nigra, red nucleus, olivary nuclei, cranial nerve nuclei. The wording “target neural circuit” as used herein can refer to neural circuits that comprise target brain cells such as trisynaptic circuit in the hippocampus.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “enhancer” refers to a type of regulatory element that can increase the efficiency of transcription, regardless of the distance or orientation of the enhancer relative to the start site of transcription.

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “variant” can refer to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “AAV” or “adeno-associated virus” refers to a Dependoparvovirus within the Parvoviridae genus of viruses. For example, the AAV can be an AAV derived from a naturally occurring “wild-type” virus, an AAV derived from a rAAV genome packaged into a capsid derived from capsid proteins encoded by a naturally occurring cap gene and/or a rAAV genome packaged into a capsid derived from capsid proteins encoded by a non-natural capsid cap gene, for example, XL.D1c-AAV9 and XL.N1-AAV9. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wild type, or naturally occurring. In some instances the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV inverted terminal repeat (ITR) sequences. The AAV genome can be a recombinant AAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV inverted terminal repeat (ITR) sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

As used herein, “native” or “wild type” can be used interchangeably, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

The term “exogenous” gene as used herein is meant to encompass all genes that do not naturally occur within the genome of an individual. For example, a miRNA could be introduced exogenously by a virus, e.g. an AAV nanoparticle.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “effective amount” refers to an amount sufficient to effect beneficial or desirable imaging, biological, diagnostic, and/or clinical results.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The term “recombinant viral vector”, as used herein, shall be given its ordinary meaning and shall also refer to a recombinant polynucleotide vector comprising one or more heterologous sequences (i.e., nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

The term “recombinant AAV vector (rAAV vector)”, as used herein, shall be given its ordinary meaning and shall also refer to a polynucleotide vector comprising one or more heterologous sequences (i.e., nucleic acid sequence not of AAV origin) that are flanked by at least one AAV ITR. Such rAAV vectors can be replicated and packaged into infectious viral particles when present in a host cell that has been infected with a suitable helper virus (or that is expressing suitable helper functions) and that is expressing AAV rep and cap gene products (i.e., AAV Rep and Cap proteins). When a rAAV vector is incorporated into a larger polynucleotide (e.g., in a chromosome or in another vector such as a plasmid used for cloning or transfection), then the rAAV vector may be referred to as a “pro-vector” which can be “rescued” by replication and encapsidation in the presence of AAV packaging functions and suitable helper functions. A rAAV vector can be in any of a number of forms, including, but not limited to, plasmids, linear artificial chromosomes, complexed with lipids, encapsulated within liposomes, and encapsidated in a viral particle, e.g., an AAV particle. A rAAV vector can be packaged into an AAV virus capsid to generate a “recombinant adeno-associated viral particle (rAAV particle)”.

The terms “rAAV virus” or “rAAV viral particle”, as used herein, shall be given their ordinary meanings and shall also refer to a viral particle composed of at least one AAV capsid protein and an encapsidated rAAV vector genome.

The term “recombinant adenoviral vector”, as used herein, shall be given its ordinary meaning and shall also refer to a polynucleotide vector comprising one or more heterologous sequences (i.e., nucleic acid sequence not of adenovirus origin) that are flanked by at least one adenovirus inverted terminal repeat sequence (ITR). In some embodiments, the recombinant nucleic acid is flanked by two inverted terminal repeat sequences (ITRs). Such recombinant viral vectors can be replicated and packaged into infectious viral particles when present in a host cell that is expressing essential adenovirus genes deleted from the recombinant viral genome (e.g., E1 genes, E2 genes. E4 genes, etc.). When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of adenovirus packaging functions. A recombinant viral vector can be in any of a number of forms, including, but not limited to, plasmids, linear artificial chromosomes, complexed with lipids, encapsulated within liposomes, and encapsidated in a viral particle, for example, an adenovirus particle. A recombinant viral vector can be packaged into an adenovirus virus capsid to generate a “recombinant adenoviral particle.”

The term “recombinant lentivirus vector”, as used herein, shall be given its ordinary meaning and shall also refer to a polynucleotide vector comprising one or more heterologous sequences (i.e., nucleic acid sequence not of lentivirus origin) that are flanked by at least one lentivirus terminal repeat sequences (LTRs). In some embodiments, the recombinant nucleic acid is flanked by two lentiviral terminal repeat sequences (LTRs). Such recombinant viral vectors can be replicated and packaged into infectious viral particles when present in a host cell that has been infected with a suitable helper functions. A recombinant lentiviral vector can be packaged into a lentivirus capsid to generate a “recombinant lentiviral particle.”

The term “recombinant herpes simplex vector (recombinant HSV vector)”, as used herein, shall be given its ordinary meaning and shall also refer to a polynucleotide vector comprising one or more heterologous sequences (i.e., nucleic acid sequence not of HSV origin) that are flanked by HSV terminal repeat sequences. Such recombinant viral vectors can be replicated and packaged into infectious viral particles when present in a host cell that has been infected with a suitable helper functions. When a recombinant viral vector is incorporated into a larger polynucleotide (e.g., in a chromosome or in another vector such as a plasmid used for cloning or transfection), then the recombinant viral vector may be referred to as a “pro-vector” which can be “rescued” by replication and encapsidation in the presence of HSV packaging functions. A recombinant viral vector can be in any of a number of forms, including, but not limited to, plasmids, linear artificial chromosomes, complexed with lipids, encapsulated within liposomes, and encapsidated in a viral particle, for example, an HSV particle. A recombinant viral vector can be packaged into an HSV capsid to generate a “recombinant herpes simplex viral particle.”

The term “heterologous” as used herein shall be given its ordinary meaning and shall also refer to an element derived from a genotypically distinct entity from that of the rest of the entity to which it is compared or into which it is introduced or incorporated. For example, a polynucleotide introduced by genetic engineering techniques into a different cell type is a heterologous polynucleotide (and, when expressed, can encode a heterologous polypeptide). Similarly, a cellular sequence (e.g., a gene or portion thereof) that is incorporated into a viral vector is a heterologous nucleotide sequence with respect to the vector.

The term “transgene”, as used herein, shall be given its ordinary meaning and shall also refer to a polynucleotide that is introduced into a cell and is capable of being transcribed into RNA and optionally, translated and/or expressed under appropriate conditions. In aspects, it confers a desired property to a cell into which it was introduced, or otherwise leads to a desired therapeutic or diagnostic outcome. In another aspect, it may be transcribed into a molecule that mediates RNA interference, such as miRNA, siRNA, or shRNA.

The term “vector genome (vg)” as used herein may refer to one or more polynucleotides comprising a set of the polynucleotide sequences of a vector, e.g., a viral vector. A vector genome may be encapsidated in a viral particle. Depending on the particular viral vector, a vector genome may comprise single-stranded DNA, double-stranded DNA, or single-stranded RNA, or double-stranded RNA. A vector genome may include endogenous sequences associated with a particular viral vector and/or any heterologous sequences inserted into a particular viral vector through recombinant techniques. For example, a recombinant AAV vector genome may include at least one ITR sequence flanking a promoter, a stuffer, a sequence of interest (e.g., an RNAi), and a polyadenylation sequence. A complete vector genome may include a complete set of the polynucleotide sequences of a vector. In some embodiments, the nucleic acid titer of a viral vector may be measured in terms of vg/mL. Methods suitable for measuring this titer are known in the art (e.g., quantitative PCR).

The terms “infection unit (iu).” “infectious particle,” or “replication unit” as used in reference to a viral titer, shall be given their ordinary meaning and shall also refer to the number of infectious and replication-competent recombinant AAV vector particles as measured by the infectious center assay, also known as replication center assay, as described, for example, in McLaughlin et al. (1988) J. Virol., 62:1963-1973.

The term “transducing unit (tu)” as used in reference to a viral titer, shall be given its ordinary meaning and shall also refer to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

An “inverted terminal repeat” or “ITR” sequence is a term well understood in the art and shall be given its ordinary meaning and shall also refer to relatively short sequences found at the termini of viral genomes which are in opposite orientation.

An “AAV inverted terminal repeat (ITR)” sequence, a term well-understood in the art, shall be given its ordinary meaning and shall also refer to an approximately 145-nucleotide sequence that is present at both termini of the native single-stranded AAV genome. The outermost 125 nucleotides of the ITR can be present in either of two alternative orientations, leading to heterogeneity between different AAV genomes and between the two ends of a single AAV genome. The outermost 125 nucleotides also contains several shorter regions of self-complementarity (designated A, A′, B, B′, C, C′ and D regions), allowing intrastrand base-pairing to occur within this portion of the ITR.

The terms “terminal resolution sequence” or “trs” shall be given their ordinary meanings and shall also refer to a sequence in the D region of the AAV ITR that is cleaved by AAV rep proteins during viral DNA replication. A mutant terminal resolution sequence is refractory to cleavage by AAV rep proteins.

The term “helper virus” for AAV, as used herein, shall be given its ordinary meaning and shall also refer to a virus that allows AAV (which is a defective parvovirus) to be replicated and packaged by a host cell. A number of such helper viruses have been identified, including adenoviruses, herpesviruses and poxviruses such as vaccinia.

### Viral Delivery of Gas Vesicle Genes

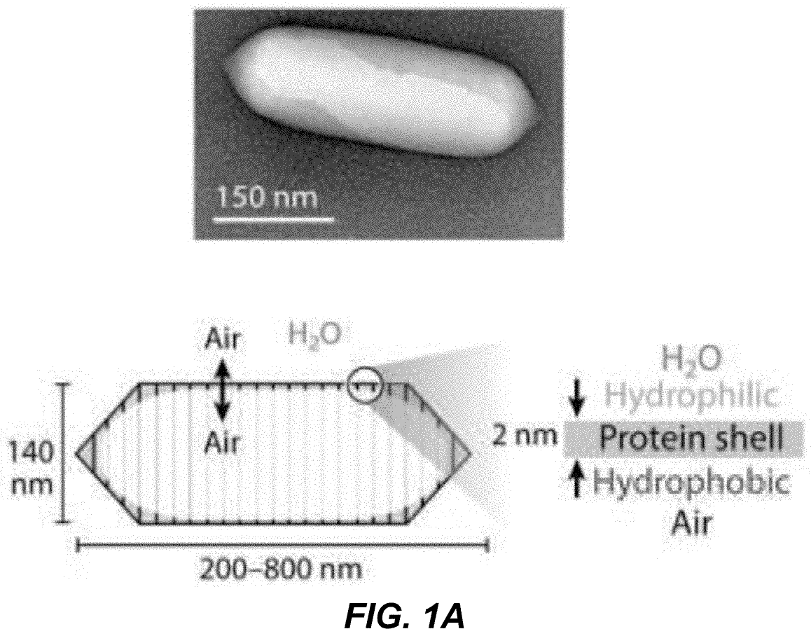

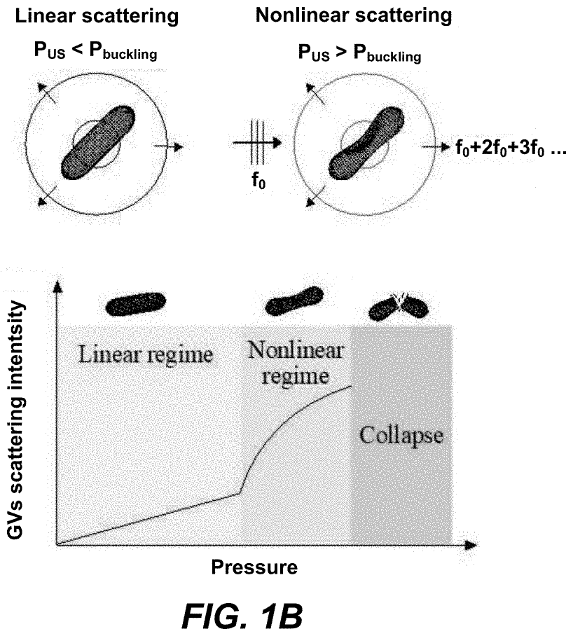

Recent work has established gas vesicles (GVs), microbially derived gas-filled protein nanostructures, as genetically encodable acoustic reporter genes. Through buckling of their exterior protein shell, GVs can produce strong nonlinear acoustic contrast that sets them apart from linear scatterers in their surrounding environment (FIGS. 1A-1B). However, the use of GVs in mammalian cells has been so far limited to transfection-compatible cell lines and the need for tedious clonal expansions of sorted cells, due to the large gene cluster size of GVs (8-14 genes). To facilitate the use of acoustic reporter genes in a broader range of biological contexts such as primary cells and endogenous tissues, there is provided, in some embodiments disclosed herein, efficient, accessible, and a versatile platforms to deliver these genes to cells both in vitro and in vivo. Disclosed herein include modular viral delivery platforms that efficiently integrate acoustic reporter genes into mammalian cells and tissues. The use of these platforms was demonstrated in a variety of contexts, including acoustic imaging of primary cells and in situ gene expression in intact mouse brain (See Example 1).

There are provided, in some embodiments, viral vector compositions. In some embodiments, the viral vector composition comprises: a single viral vector comprising one or more first promoters operably connected to one or more gas vesicle (GV) polynucleotides comprising: one or more gas vesicle assembly (GVA) gene(s) encoding one or more GVA protein(s), and one or more gas vesicle structural (GVS) gene(s) encoding one or more GVS protein(s), wherein the one or more GVA protein(s) and the one or more GVS protein(s) are capable of forming gas vesicles (GVs) (e.g., a plurality of GVs) upon expression in a cell.

There are provided, in some embodiments, viral vector compositions. In some embodiments, the viral vector composition comprises: (i) a first viral vector comprising one or more first promoters operably connected to one or more gas vesicle (GV) polynucleotides comprising: one or more gas vesicle structural (GVS) gene(s) encoding one or more GVS protein(s); and (ii) a second viral vector comprising one or more first promoters operably connected to one or more GV polynucleotides comprising: one or more gas vesicle assembly (GVA) gene(s) encoding one or more GVA protein(s), wherein the one or more GVA protein(s) and the one or more GVS protein(s) are capable of forming gas vesicles (GVs) (e.g., a plurality of GVs) upon expression in a cell. The first viral vector can comprise one or more first promoters operably connected to one or more gas vesicle (GV) polynucleotides comprising: one or more gas vesicle assembly (GVA) gene(s) encoding one or more GVA protein(s).

There are provided, in some embodiments, viral vector compositions. In some embodiments, the viral vector composition comprises: (i) a primary viral vector comprising one or more first promoters operably connected to one or more gas vesicle (GV) polynucleotides comprising: one or more gas vesicle structural (GVS) gene(s) encoding one or more GVS protein(s); (ii) a secondary viral vector comprising one or more first promoters operably connected to one or more GV polynucleotides comprising: one or more gas vesicle assembly (GVA) gene(s) encoding one or more GVA protein(s); and (iii) a tertiary viral vector comprising one or more first promoters operably connected to one or more GV polynucleotides comprising: one or more gas vesicle assembly (GVA) gene(s) encoding one or more GVA protein(s), wherein the one or more GVA protein(s) and the one or more GVS protein(s) are capable of forming gas vesicles (GVs) (e.g., a plurality of GVs) upon expression in a cell. The primary viral vector can comprise one or more first promoters operably connected to one or more gas vesicle (GV) polynucleotides comprising: one or more gas vesicle assembly (GVA) gene(s) encoding one or more GVA protein(s).

The viral vector composition can comprise a single viral vector, a first viral vector, a second viral vector, a primary viral vector, a secondary viral vector, a tertiary viral vector, or any combination thereof, and each of these can be derived from the same or different viral species. The viral vector (e.g., a single viral vector, a first viral vector, a second viral vector, a primary viral vector, a secondary viral vector, a tertiary viral vector) can be a recombinant viral vector and/or can be encapsidated in a viral particle. A viral vector (e.g., a single viral vector, a first viral vector, a second viral vector, a primary viral vector, a secondary viral vector, a tertiary viral vector) can comprise a polynucleotide encoding a detectable protein. The detectable protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, mScarlet, derivatives thereof, or any combination thereof.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The promoter (e.g., first promoter, context-dependent promoter) can vary in length, for example be less than 1 kb. In other embodiments, the promoter (e.g., first promoter, context-dependent promoter) is greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter may provide expression of the transactivator, GVA protein(s), and/or GVS protein(s) for a period of time in targeted tissues such as, but not limited to, the CNS. Expression of the transactivator, GVA protein(s), and/or GVS protein(s) can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

The degree of gene expression in the target cell can vary. The amount of the transactivator, GVA protein(s), and/or GVS protein(s) expressed in the subject (e.g., the target brain region(s) of the subject) can vary. For example, in some embodiments the transactivator, GVA protein(s), and/or GVS protein(s) can be expressed in the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the transactivator, GVA protein(s), and/or GVS protein(s) are expressed in the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a protein is needed for the method to be effective can vary, and an effective amount of GV expression can be readily determined by a skilled artisan using conventional methods known in the art without undue experimentation.

In some embodiments, a viral vector (e.g., a single viral vector, a first viral vector, a second viral vector, a primary viral vector, a secondary viral vector, a tertiary viral vector) comprises: a context-dependent promoter operably linked to a transactivator polynucleotide comprising a transactivator gene. The context-dependent promoter can be capable of inducing transcription of the transactivator gene to generate a transactivator transcript. The transactivator transcript can be capable of being translated to generate a transactivator. The activity of the context-dependent promoter and/or the degree of expression of the transactivator can be associated with the presence and/or amount a unique cell type and/or a unique cell state. In some embodiments, in the presence of the transactivator and a transactivator-binding compound, the first promoter is capable of inducing transcription of the one or more GV polynucleotides to generate GV transcript(s). The GV transcript(s) can be capable of being translated to generate GVA protein(s) and/or GVS protein(s). The first promoter can comprise one or more copies of a transactivator recognition sequence the transactivator is capable of binding to induce transcription, and the transactivator can be incapable of binding the transactivator recognition sequence in the absence of the transactivator-binding compound. The one or more copies of a transactivator recognition sequence can comprise one or more copies of a tet operator (TetO). The first promoter can comprise a tetracycline response element (TRE), and the TRE can comprise one or more copies of a tet operator (TetO). The transactivator can comprise reverse tetracycline-controlled transactivator (rtTA). The transactivator can comprise tetracycline-controlled transactivator (tTA). The transactivator-binding compound can comprise tetracycline, doxycycline or a derivative thereof. The transactivator can comprise a constitutive signal peptide for protein degradation (e.g., PEST).

The degree of expression of the transactivator can be positively correlated with the presence and/or amount a unique cell type and/or a unique cell state. A unique cell type and/or a unique cell state can comprise a unique gene expression pattern. The unique cell type and/or unique cell state can comprise a unique anatomic location. The unique cell type and/or the unique cell state can comprise anatomically locally unique gene expression. The context-dependent promoter can be an Immediate Early Gene (IEG) regulated promoter (e.g., cFos, pRAM, or EGR1). The unique cell state can comprise activation of one or more cellular activities of interest, such as, for example, cellular activities associated with neural activity (e.g., fear conditioning, memory formation, learning, and/or sensory modalities). A unique cell type and/or a unique cell state can be caused by hereditable, environmental, and/or idiopathic factors. A unique cell type and/or a unique cell state can be caused by and/or associated with the expression of one or more endogenous proteins whose expression is regulated by the endogenous version of the context-dependent promoter. The unique cell state and/or unique cell type can be characterized by signaling of one or more endogenous signal transducer(s) (e.g., signal transducer(s) regulated by the endogenous context-dependent promoter). In some embodiments, the unique cell type and/or the cell in the unique cell state (i) causes and/or aggravates a disease or disorder and/or (ii) is associated with the pathology of a disease or disorder. The expression of the GVs (and the detection of the nonlinear contrast generated by said expressed GVs) can enable imaging of in situ gene expression. Said GVs can serve as a proxy for gene expression of interest (e.g., a unique cell type, a unique cell state, expression of a gene product under the control of the endogenous version of the context-dependent promoter).

In some embodiments, the unique cell state comprises: a physiological state (e.g., a cell cycle state, a differentiation state, a development state, a metabolic state, or a combination thereof); and/or a pathological state (e.g., a disease state, a human disease state, a diabetic state, an immune disorder state, a neurodegenerative disorder state, an oncogenic state, or a combination thereof). The unique cell state and/or unique cell type can be characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

The unique cell state and/or unique cell type can be characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression.

The unique cell state and/or unique cell type can be characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion. In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The context-dependent promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a hSynapsin promoter, a α-myosin heavy chain (a-MHC) promoter, or a cardiac Troponin T (cTnT) promoter. The tissue specific promoter can be a neuronal activity-dependent promoter and/or a neuron-specific promoter (e.g., a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ 1.7 promoter, a Naᵥ 1.8 promoter, a Naᵥ 1.9 promoter, or an Advillin promoter). The tissue specific promoter can be a muscle-specific promoter. The muscle-specific promoter can comprise a creatine kinase (MCK) promoter. The context-dependent promoter can comprise or can be derived from an endogenous promoter associated with a unique cell type and/or unique cell state. The context-dependent promoter can be a neuron cell specific promoter such as, for example, a tyrosine hydroxylase promoter, a melanopsin promoter, or a promoter that expresses in retinal neurons. In another embodiment, the neuron cell specific promoter can be a PRSx8 promoter which specifically targets catecholaminergic neurons. PRSx8 is based on an upstream regulatory site in the human Dopamine Beta-Hydroxylase (“DBH”) promoter and drives high levels of expression in adrenergic neurons. In another embodiment, the neuron-specific promoter can be preprotachykinin-1 promoter (TAC-1). Other exemplary context-dependent promoters include neuron-specific enolase (NSE) and the promoters listed in the following Table 1.

TABLE 1
Exemplary Context-dependent Promoters

| Name | Size | | Specificity |
|---|---|---|---|
| GFAP104 | 845 | bp | Hybrid of EF1a and GFAP |
| CamKlla | 1.2 | kb | Specific expression in excitatory neurons |
| | | | in the neocortex and hippocampus |
| CK0.4 | 217 | bp | Calcium/Calmodulin-dependent kinase II alpha |
| GFAP | 2.0 | kb | Specific in astrocyte |
| MBP | 1.3 | kb | Myelin basic protein promoter, efficient |
| | | | transduction of oligodendrocytes by adeno- |
| | | | associated virus type 8 vectors |
| Synapsin | 471 | bp | Specific in neuron |
| Mecp2 | 230 | bp | Truncated Mcep2 neuron specific |
| c-fos | 1.7 | kb | Activity-dependent promoter |
| Somatostat | 1.2 | kb | Restricting expression to GABAergic neuron |
| Rpe65 | 700 | bp | Retinal Pigment epithelium-specific |
| | | | expression in vivo and in vitro |
| NSE | 1.3 | kb | Neuron-specific enolase promoter |

Provided herein are viral vectors (e.g., a single viral vector, a first viral vector, a second viral vector, a primary viral vector, a secondary viral vector, a tertiary viral vector) encoding gas-filled protein structures (GVPS), also referred to as “gas vesicles” (GVs). The phrases “gas vesicles protein structure” or “GV”, “GVP”, “GVPS” or “Gas Vesicles” as used herein shall be given their ordinary meaning, and shall also refer to a gas-filled protein structure intracellularly expressed by certain bacteria or archea as a mechanism to regulate cellular buoyancy in aqueous environments. GVs are described in Walsby, A. E. ((1994). Gas vesicles. Microbiology and Molecular Biology Reviews, 58(1), 94-144) hereby incorporated by reference in its entirety. The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as gvpA/B) and optionally also a GVS identified as gvpC. The compositions, methods and systems described herein can be used with compositions, methods and systems (e.g., gas vesicle compositions and ultrasonic methods) previously described in U.S. Patent Application Publication Nos. 2014/0288411, 2014/0288421, 2018/0030501, 2018/0038922, 2019/0175763, 2019/0314001, 2020/0164095, 2020/0237346, and International Patent Application Publication WO2020/146379; the content of each of these applications is incorporated herein by reference in its entirety.

In particular, a GV in the sense of the disclosure is a structure intracellularly expressed by bacteria or archaea forming a hollow structure wherein a gas is enclosed by a protein shell, which is a shell substantially made of protein (up at least 95% protein). In gas vesicles in the sense of the disclosure, the protein shell is formed by a plurality of proteins herein also indicated as Gyp proteins or Gvps, which are expressed by the bacteria or archaea and form in the bacteria or archaea cytoplasm a gas permeable and liquid impermeable protein shell configuration encircling gas. Accordingly, a protein shell of a GV is permeable to gas but not to surrounding liquid such as water. For example, GVs' protein shells exclude liquid water but permit gas to freely diffuse in and out from the surrounding media making them physically stable despite their usual nanometer size.

GV structures are typically nanostructures with widths and lengths of nanometer dimensions (in particular with widths of 45-250 nm and lengths of 100-800 nm) but can have lengths as large as the dimensions of a cell in which they are expressed, as will be understood by a skilled person. GVs and methods are described in Farhadi et al, Science, 2019, hereby incorporated by reference. In certain embodiments, the gas vesicles protein structure have average dimensions of 1000 nm or less, such as 900 nm or less, including 800 nm or less, or 700 nm or less, or 600 nm or less, or 500 nm or less, or 400 nm or less, or 300 nm or less, or 250 nm or less, or 200 nm or less, or 150 nm or less, or 100 nm or less, or 75 nm or less, or 50 nm or less. For example, the average diameter of the gas vesicles may range from 10 nm to 1000 nm, such as 25 nm to 500 nm, including 50 nm to 250 nm, or 100 nm to 250 nm. By “average” is meant the arithmetic mean.

GVs in the sense of the disclosure have different shapes depending on their genetic origins. For example, GVs in the sense of the disclosure can be substantially spherical, ellipsoid, cylindrical, or have other shapes such as football shape or cylindrical with cone shaped end portions depending on the type of bacteria providing the gas vesicles.

The term Gas Vesicle Structural (GVS) proteins as used herein indicates proteins forming part of a gas-filled protein structure intracellularly expressed by certain bacteria or archaea and can be used as a mechanism to regulate cellular buoyancy in aqueous environments. In particular, GVS shell comprises a GVS identified as gvpA or gvpB (herein also referred to as Gyp A/B) and optionally also a GVS identified as gvpC. GvpA is a structural protein that assembles through repeated unites to make up the bulk of GVs. GvpC is a scaffold protein with 5 repeat units that assemble on the outer shell of GVs. GvpC can be engineered to tune the mechanical and acoustic properties of GVs as well as act as a handle for appending moieties on to. A gvpC protein is a hydrophilic protein of a GV shell, which includes repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another.

The optional gvpC gene encodes for a gvpC protein which is a hydrophilic protein of a GV shell, including repetitions of one repeat region flanked by an N-terminal region and a C terminal region. The term “repeat region” or “repeat” as used herein with reference to a protein can refer to the minimum sequence that is present within the protein in multiple repetitions along the protein sequence without any gaps. Accordingly, in a gvpC multiple repetitions of a same repeat is flanked by an N-terminal region and a C-terminal region. In a same gvpC, repetitions of a same repeat in the gvpC protein can have different lengths and different sequence identity one with respect to another. In performing alignment steps sequence are identified as repeat when the sequence shows at least 3 or more of the characteristics described in U.S. application Ser. No. 15/663,635 published as US 2018/0030501 (incorporated herein by reference in its entirety) which also include additional features of gvpC proteins and the related identification.

The phrase “GV type” as used herein shall be given its ordinary meaning, and shall also refer to a gas vesicle having dimensions and shape resulting in distinctive mechanical, acoustic, surface and/or magnetic properties as will be understood by a skilled person upon reading of the present disclosure. In particular, a skilled person will understand that different shapes and dimensions will result in different properties in view of the indications in provided in U.S. application Ser. No. 15/613,104 and U.S. Ser. No. 15/663,600 and additional indications identifiable by a skilled person. In some embodiments, the nucleic acid compositions provided herein encode a combination of different GV types and/or variants thereof, with each expressed GV exhibiting a different acoustic collapse profile with progressively decreased midpoint collapse pressure values. In some embodiments, the percentage difference between the midpoint collapse pressure values of any given two expressed GVs types is at least twenty percent.

In some embodiments, GVs are capable of withstanding pressures of several kPa. but collapse irreversibly at a pressure at which the GV protein shell is deformed to the point where it flattens or breaks, allowing the gas inside the GV to dissolve irreversibly in surrounding media, herein also referred to as a critical collapse pressure, or selectable critical collapse pressure, as there are various points along a collapse pressure profile (e.g., peak acoustic pressure).

A collapse pressure profile (e.g., peak acoustic pressure) as used herein indicates a range of pressures over which collapse of a population of GVs of a certain type occurs. In particular, a collapse pressure profile in the sense of the disclosure comprise increasing acoustic collapse pressure values, starting from an initial collapse pressure value at which the GV signal/optical scattering by GVs starts to be erased to a complete collapse pressure value at which the GV signal/optical scattering by GVs is completely erased. The collapse pressure profile of a set type of GV is thus characterized by a mid-point pressure where 50% of the GVs of the set type have been collapsed (also known as the “midpoint collapse pressure”), an initial collapse pressure where 5% or lower of the GVs of the type have been collapsed, and a complete collapse pressure where at least 95% of the GVs of the type have been collapsed. In some embodiments herein described a selectable critical collapse pressure can be any of these collapse pressures within a collapse pressure profile, as well as any point between them. The critical collapse pressure profile of a GV is functional to the mechanical properties of the protein shell and the diameter of the shell structure. U.S. Patent Application Publication No. 2020/0164095 describes gas vesicles, protein variants and related compositions methods and systems for singleplexed and/or multiplexed ultrasonic methods (e.g., imaging of a target site in which a gas vesicle provides contrast for the imaging) which is modifiable by application of a selectable acoustic collapse pressure value of the gas vesicle, the content of which is hereby expressly incorporated by reference in its entirety.

The acoustic collapse pressure profile (e.g., peak acoustic pressure) of a given GV type can be determined by imaging GVs with imaging ultrasound energy after collapsing portions of the given GV type population with a collapsing ultrasound energy (e.g. ultrasound pulses) with increasing peak positive pressure amplitudes to obtain acoustic pressure data point of acoustic pressure values, the data points forming an acoustic collapse curve. The acoustic collapse pressure function f(p) can be derived from the acoustic collapse curve by fitting the data with a sigmoid function such as a Boltzmann sigmoid function. An acoustic collapse pressure profile in the sense of the disclosure can include a set of initial collapse pressure values, a midpoint collapse pressure value and a set of complete collapse pressure values. The initial collapse pressures are the acoustic collapse pressures at which 5% or less of the GV signal is erased. A midpoint collapse pressure is the acoustic collapse pressure at which 50% of the GV signal is erased. Complete collapse pressures are the acoustic collapse pressures at which 95% or more of the GV signal is erased. The pressure can be peak pressure. In some embodiments, the peak pressure is peak positive pressure. In some embodiments, the peak pressure is peak negative pressure.

U.S. Patent Application Publication No. 2018/0030501 describes hybrid gas vesicle gene cluster (GVGC) configured for expression in a prokaryotic host comprising gas vesicle assembly (GVA) genes native to a GVA prokaryotic species and capable of being expressed in a functional form in the prokaryotic host, as well as one or more gas vesicle structural (GVS) genes native to one or more GVS prokaryotic species, at least one of the one or more GVS prokaryotic species different from the GVA prokaryotic species, and related gas vesicle reporting (GVR) genetic circuits, genetic, vectors, engineered cells, and related compositions methods and systems to produce GVs, hybrid GVGC and/or image a target site, the content of which is hereby expressly incorporated by reference in its entirety. The term “Gas Vesicle Genes Cluster” or “GVGC” as described herein indicates a gene cluster encoding a set of GV proteins capable of providing a GV upon expression within a cell. In some embodiments, the nucleic acid compositions provided herein encode some or all elements of a GVGC. The term “gene cluster” as used herein means a group of two or more genes found within an organism's DNA that encode two or more polypeptides or proteins, which collectively share a generalized function or are genetically regulated together to produce a cellular structure and are often located within a few thousand base pairs of each other. The size of gene clusters can vary significantly, from a few genes to several hundred genes. Portions of the DNA sequence of each gene within a gene cluster are sometimes found to be similar or identical; however, the resulting protein of each gene is distinctive from the resulting protein of another gene within the cluster. Genes found in a gene cluster can be observed near one another on the same chromosome or native plasmid DNA, or on different, but homologous chromosomes. An example of a gene cluster is the Hox gene, which is made up of eight genes and is part of the Homeobox gene family. In the sense of the disclosure, gene clusters as described herein also comprise gas vesicle gene clusters, wherein the expressed proteins thereof together are able to form gas vesicles.

Engineered GVs and methods of tuning the acoustic properties thereof are provided in U.S. Patent Application Publication No. 2020/0164095, the content of which is incorporated herein by reference in its entirety. In some embodiments, the GVs can be engineered to modulate the GV mechanical, acoustic, surface and targeting properties in order to achieve enhanced harmonic responses and multiplexed imaging to be better distinguished from background tissues. In some embodiments herein described Gas vesicles protein structures can be provided by Gyp genes endogenously expressed in bacteria or archaea. Endogenous expression can refer to expression of Gyp proteins forming the protein shell of the GV in bacteria or archaea that naturally produce gas vesicles encoded (e.g. in their genome or native plasmid DNA). Gyp proteins expressed by bacteria or archaea typically include two primary structural proteins, here also indicated as GvpA and GvpC, and several putative minor components and chaperones as would be understood by a person skilled in the art. In some embodiments, heterologously expressed Gyp proteins to provide a GV type have independently at least 50% sequence identity, preferably at least 80%, more preferably at least 90%, most preferably at least 95% sequence identity compared to a reference sequence of corresponding Gyp protein using one of the alignment programs described using standard parameters.

The GVA genes and/or GVS genes can be derived from Bacillus Megaterium, Anabaena flos-aquae, Serratia sp., Bukholderia thailandensis, B. megaterium, Frankia sp, Haloferax mediaterranei, Halobacterium sp, Halorubrum vacuolatum, Microcystis aeruginosa, Methanosarcina barkeri, Streptomyces coelicolor, and/or Psychromonas ingrahamii. In some embodiments, the one or more GV polynucleotides comprise: two or more GVS genes derived from different prokaryotic species; GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii; gvpB, gvpN gvpF, gvpG, gvpL gvpS, gvpK, gvpJ, and/or gvpU from B. megaterium; gvpA, gvpC, gvpN, gvpJ, gvpK, gvpF, gvpG, gvpV, and/or gvpW from Anabaena flos-aquae; gvpR, gvpN, gvpF, gvpG, gvpL, gvpS, gvpK, gvpJ, gvpT and/or gvpU from B. megaterium and gvpA from Anabaena flos-aquae; gvpA, and/or gvpC from Anabaena flos-aquae, and gvpN, gvpF, gvpG, gvpL, gvpS, gvpK, gvpJ, and/or gvpU from B. megaterium; and/or gvpA, gvpC and/or gvpN from Anabaena flos-aquae, and gvpF, gvpG, gvpL, gvpS, gvpK, gvpJ, and/or gvpU from B. megaterium. In some embodiments, the GVA genes and GVS genes have sequences codon optimized for expression in a eukaryotic cell. The GVs can comprise a GVS variant engineered to present a tag enabling clustering in the cell. The GVs can be hybrid GVs derived from two or more prokaryotic species.

The plurality of gas vesicles can comprise a first collapse pressure profile. The first collapse pressure profile can comprise a collapse function from which a gas vesicle collapse amount can be determined for a given pressure value. The first collapse pressure profile can comprise a first initial collapse pressure where 5% or lower of the plurality of gas vesicles are collapsed, a first midpoint collapse pressure where 50% of the plurality of gas vesicles are collapsed, a first complete collapse pressure where at least 95% of the plurality of gas vesicles are collapsed, any pressure between the first initial collapse pressure and the first midpoint collapse pressure, and any pressure between the first midpoint collapse pressure and the first complete collapse pressure. In some embodiments, a first selectable collapse pressure is: any collapse pressure within the first collapse pressure profile; selected from the first collapse pressure profile at a value between 0.05% collapse of the plurality of gas vesicles and 95% collapse of the plurality of gas vesicles; equal to or greater than the first initial collapse pressure; equal to or greater than the first midpoint collapse pressure; and/or equal to or greater than the first complete collapse pressure.

The plurality of gas vesicles can comprise a first buckling pressure profile. The first collapse pressure profile can comprise a buckling function from which a gas vesicle buckling amount can be determined for a given pressure value. The buckling amount can comprise the amount of nonlinear contrast. The first buckling pressure profile can comprise a first buckling threshold pressure where the gas vesicles starts to buckle and produce nonlinear contrast, a first optimum buckling pressure where the gas vesicles exhibit maximum buckling and produce the highest level of nonlinear contrast, a first collapse pressure wherein the gas vesicles collapse, any pressure between the first buckling threshold pressure and the first optimum buckling pressure, and any pressure between the first optimum buckling pressure and the first collapse pressure. In some embodiments, a first selectable buckling pressure is: any collapse pressure within the first buckling pressure profile; the first optimum buckling pressure; and/or equal to or less than the first initial collapse pressure.

In some embodiments, the single viral vector comprises: a first GV polynucleotide encoding GvpA, a second GV polynucleotide encoding GvpN, a third GV polynucleotide encoding GvpJ, a fourth GV polynucleotide encoding GvpK, a fifth GV polynucleotide encoding GvpF, a sixth GV polynucleotide encoding GvpG, a seventh GV polynucleotide encoding GvpW, and an eighth GV polynucleotide encoding GvpV. Each of the GV polynucleotides can be operably connected to a tandem gene expression element.

In some embodiments, the first viral vector comprises: a first GV polynucleotide encoding GvpA. In some embodiments, the second viral vector comprises: a second GV polynucleotide encoding GvpN, a third GV polynucleotide encoding GvpJ, a fourth GV polynucleotide encoding GvpK, a fifth GV polynucleotide encoding GvpF, a sixth GV polynucleotide encoding GvpG, a seventh GV polynucleotide encoding GvpW, and an eighth GV polynucleotide encoding GvpV. Each of the GV polynucleotides can be operably connected to a tandem gene expression element.

In some embodiments, the primary viral vector comprises: a first GV polynucleotide encoding GvpA. In some embodiments, the secondary viral vector comprises: a second GV polynucleotide encoding GvpG, a third GV polynucleotide encoding GvpW, and a fourth GV polynucleotide encoding GvpV. In some embodiments, the tertiary viral vector comprises: a fifth GV polynucleotide encoding GvpN, a sixth GV polynucleotide encoding GvpJ, a seventh GV polynucleotide encoding GvpK, and an eighth GV polynucleotide encoding GvpF. Each of the GV polynucleotides can be operably connected to a tandem gene expression element.

In some embodiments, one or more GV polynucleotides and/or transactivator polynucleotide comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the one or more first promoters comprise: a minimal promoter (e.g., TATA, miniCMV, and/or miniPromo); a tissue-specific promoter and/or a lineage-specific promoter; and/or a ubiquitous promoter (e.g., a minEf1a promoter, a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

The systems, methods, compositions, and kits provided herein can, in some embodiments, be employed in concert with the systems, methods, compositions, and kits comprising recombinant adeno-associated virus (rAAV) comprising an AAV acoustic targeting peptide exhibiting increased transduction at site(s) of focused ultrasound blood-brain barrier opening (FUS-BBBO), increased neuronal tropism, and diminished transduction of peripheral organs described in U.S. patent application Ser. No. 17/814,384, entitled, “VIRAL VECTORS FOR ENHANCED ULTRASOUND-MEDIATED DELIVERY TO THE BRAIN,” filed Jul. 22, 2022, the content of which is incorporated herein by reference in its entirety.

GV production requires the co-expression of multiple GV proteins (Gvps), which in prokaryotes are expressed from polycistronic operons at specific ratios determined by the strength of their respective ribosome binding sites or other regulatory mechanisms. The systems, methods, compositions, and kits provided herein can, in some embodiments, be employed in concert with the systems, methods, compositions, and kits expression of multiple proteins from a single mRNA with a predetermined stoichiometry described in U.S. patent application Ser. No. 17/866,240, entitled “STOICHIOMETRIC EXPRESSION OF MESSENGER POLYCISTRONS”, filed Jul. 15, 2022, the content of which is incorporated herein by reference in its entirety.

The viral vector (e.g., a single viral vector, a first viral vector, a second viral vector, a primary viral vector, a secondary viral vector, a tertiary viral vector) can be, can comprise, or can be derived from, an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector derivatives thereof, or any combination thereof. The viral vector can be capable of integrating into a mammalian cell genome, and the GVs can be capable of being expressed upon integration.

In some embodiments, the viral vector is a recombinant lentiviral vector. In some embodiments, the viral particle is a lentiviral particle. Lentiviruses are positive-sense, ssRNA retroviruses with a genome of approximately 10 kb. Lentiviruses are known to integrate into the genome of dividing and non-dividing cells. Lentiviral particles may be produced, for example, by transfecting multiple plasmids (typically the lentiviral genome and the genes required for replication and/or packaging are separated to prevent viral replication) into a packaging cell line, which packages the modified lentiviral genome into lentiviral particles. In some embodiments, a lentiviral particle may refer to a first generation vector that lacks the envelope protein. In some embodiments, a lentiviral particle may refer to a second generation vector that lacks all genes except the gag/pol and tat/rev regions. In some embodiments, a lentiviral particle may refer to a third generation vector that only contains the endogenous rev, gag, and pol genes and has a chimeric LTR for transduction without the tat gene (see Dull. T. et al. (1998) J. Virol. 72:8463-71). For further description, see Durand, S. and Cimarelli, A. (2011) Viruses 3:132-59.

Use of any lentiviral vector is considered within the scope of the disclosed compositions and methods. In some embodiments, the lentiviral vector is derived from a lentivirus including, without limitation, human immunodeficiency virus-1 (HIV-1), human immunodeficiency virus-2 (HIV-2), simian immunodeficiency virus (SIV), feline immunodeficiency virus (FIV), equine infectious anemia virus (EIAV), bovine immunodeficiency virus (BIV), Jembrana disease virus (JDV), visna virus (VV), and caprine arthritis encephalitis virus (CAEV).

In some embodiments, the viral vector is encapsidated in a viral particle. In some embodiments, the viral particle is a recombinant lentiviral particle encapsidating a recombinant lentiviral vector. In some embodiments, the recombinant viral particles comprise a lentivirus vector in combination with one or more foreign viral capsid proteins. Such combinations may be referred to as pseudotyped recombinant lentiviral particles. In some embodiments, foreign viral capsid proteins used in pseudotyped recombinant lentiviral particles are derived from a foreign virus. In some embodiments, the foreign viral capsid protein used in pseudotyped recombinant lentiviral particles is Vesicular stomatitis virus glycoprotein (VSV-GP). VSV-GP interacts with a ubiquitous cell receptor, providing broad tissue tropism to pseudotyped recombinant lentiviral particles. In addition, VSV-GP is thought to provide higher stability to pseudotyped recombinant lentiviral particles. In other embodiments, the foreign viral capsid proteins are derived from, including without limitation. Chandipura virus. Rabies virus. Mokola virus, Lymphocytic choriomeningitis virus (LCMV). Ross River virus (RRV), Sindbis virus, Semliki Forest virus (SFV), Venezuelan equine encephalitis virus. Ebola virus Reston, Ebola virus Zaire, Marburg virus, Lassa virus, Avian leukosis virus (ALV), Jaagsiekte sheep retrovirus (JSRV). Moloney Murine leukemia virus (MLV). Gibbon ape leukemia virus (GALV). Feline endogenous retrovirus (RD114). Human T-lymphotropic virus 1 (HTLV-1), Human foamy virus, Maedi-visna virus (MVV), SARS-CoV, Sendai virus, Respiratory syncytia virus (RSV), Human parainfluenza virus type 3, Hepatitis C virus (HCV), Influenza virus. Fowl plague virus (FPV), or Autographa californica multiple nucleopolyhedro virus (AcMNPV).

In some embodiments, the recombinant lentiviral vector is derived from a lentivirus pseudotyped with vesicular stomatitis virus (VSV), lymphocytic choriomeningitis virus (LCMV), Ross river virus (RRV), Ebola virus. Marburg virus, Mokala virus, Rabies virus, RD114, or variants therein. Examples of vector and capsid protein combinations used in pseudotyped Lentivirus particles can be found, for example, in Cronin. J. et al. (2005). Curr. Gene Ther. 5(4):387-398. Different pseudotyped recombinant lentiviral particles can be used to optimize transduction of particular target cells or to target specific cell types within a particular target tissue (e.g., a diseased tissue). For example, tissues targeted by specific pseudotyped recombinant lentiviral particles, include without limitation, liver (e.g. pseudotyped with a VSV-G, LCMV, RRV, or SeV F protein), lung (e.g, pseudotyped with an Ebola, Marburg, SeV F and HN, or JSRV protein), pancreatic islet cells (e.g, pseudotyped with an LCMV protein), central nervous system (e.g, pseudotyped with a VSV-G. LCMV. Rabies, or Mokola protein), retina (e.g, pseudotyped with a VSV-G or Mokola protein), monocytes or muscle (e.g, pseudotyped with a Mokola or Ebola protein), hematopoietic system (e.g, pseudotyped with an RD114 or GALV protein), or cancer cells (e.g, pseudotyped with a GALV or LCMV protein). For further description, see Cronin, J. et al. (2005). Curr. Gene Ther. 5(4):387-398 and Kay, M. et al. (2001) Nat. Med. 7(1):33-40. In some embodiments, the recombinant lentiviral particle comprises a capsid pseudotyped with vesicular stomatitis virus (VSV), lymphocytic choriomeningitis virus (LCMV). Ross river virus (RRV). Ebola virus, Marburg virus. Mokala virus, Rabies virus. RD114 or variants therein.

A viral vector can be a lentiviral vector (e.g., human immunodeficiency virus 1 (HIV-1), human immunodeficiency virus 2 (HIV-2), visna-maedi virus (VMV) virus, caprine arthritis-encephalitis virus (CAEV), equine infectious anemia virus (EIAV), feline immunodeficiency virus (FIV), bovine immune deficiency virus (BIV), simian immunodeficiency virus (SIV), derivatives thereof, or any combination thereof). A viral vector can be a recombinant lentiviral vector. The recombinant lentiviral vector can be derived from a lentivirus pseudotyped with vesicular stomatitis virus (VSV), lymphocytic choriomeningitis virus (LCMV), Ross river virus (RRV), Ebola virus, Marburg virus, Mokala virus, Rabies virus, RD114, or variants therein. The viral vector composition can comprise: one or more of a left (5′) retroviral LTR, a Psi (Ψ) packaging signal, a central polypurine tract/DNA flap (cPPT/FLAP), a retroviral export element, and a right (3′) retroviral LTR. The promoter of the 5′ LTR can be replaced with a heterologous promoter. The 5′ LTR or 3′ LTR can be a lentivirus LTR. The 3′ LTR can comprise one or more modifications and/or deletions. The 3′ LTR can be a self-inactivating (SIN) LTR.

In some embodiments, the viral vector is a recombinant adenoviral vector. In some embodiments, the viral particle is an adenoviral particle. In some embodiments, the adenoviral particle is a recombinant adenoviral particle. In some embodiments, the adenoviral particle lacks or contains a defective copy of one or more E1 genes, which renders the adenovirus replication-defective. Adenoviruses include a linear, double-stranded DNA genome within a large (˜950 Å), non-enveloped icosahedral capsid. Adenoviruses have a large genome that can incorporate more than 30 kb of heterologous sequence (e.g., in place of the E1 and/or E3 region), making them uniquely suited for use with larger heterologous genes. They are also known to infect dividing and non-dividing cells and do not naturally integrate into the host genome (although hybrid variants may possess this ability). In some embodiments, the adenoviral vector may be a first generation adenoviral vector with a heterologous sequence in place of E1. In some embodiments, the adenoviral vector may be a second generation adenoviral vector with additional mutations or deletions in E2A. E2B, and/or E4. In some embodiments, the adenoviral vector may be a third generation or gutted adenoviral vector that lacks all viral coding genes, retaining only the ITRs and packaging signal and requiring a helper adenovirus in trans for replication, and packaging. Adenoviral particles have been investigated for use as vectors for transient transfection of mammalian cells as well as gene therapy vectors. For further description, see. e.g., Danthinne. X, and Imperiale, M. J. (2000) Gene Ther. 7:1707-14 and Tatsis. N, and Ertl. H. C. (2004)Mol. Ther. 10:616-29.

In some embodiments, the viral vector is an rHSV vector. In some embodiments, the viral particle is a herpes simplex virus (HSV) particle. In some embodiments, the HSV particle is a rHSV particle. HSV is an enveloped, double-stranded DNA virus with a genome of approximately 152 kb. Advantageously, approximately half of its genes are nonessential and may be deleted to accommodate heterologous sequence. HSV particles infect non-dividing cells. In addition, they naturally establish latency in neurons, travel by retrograde transport, and can be transferred across synapses, making them advantageous for transfection of neurons and/or gene therapy approaches involving the nervous system. In some embodiments, the HSV particle may be replication-defective or replication-competent (e.g., competent for a single replication cycle through inactivation of one or more late genes). For further description, see Manservigi, R. et al. (2010) Open Virol. J. 4:123-56.

The AAV vector can comprise single-stranded AAV (ssAAV) vector or a self-complementary AAV (scAAV) vector. The AAV vector can comprise AAV1, AAV2, AAV3, AAV4, AAV5, AAV6, AAV7, AAV8, AAV9, derivatives thereof, or any combination thereof. The AAV vector can comprise an AAV9 variant engineered for systemic delivery (e.g., AAV-PHP.B, AAV-PHP.eB, or AAV-PHP.S). The AAV vector can be or can comprise an AAV selected from the group consisting of AAV9, AAV9 K449R (or K449R AAV9), AAV1, AAVrhlO, AAV-DJ, AAV-DJ8, AAV5, AAVPHP.B (PHP.B), AAVPHP.A (PUPA), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2 Al 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAVl-7/rh.48, AAVl-8/rh.49, AAV2-15/rh.62, AAV2-3/rh.61, AAV2-4/rh.50, AAV2-5/rh.51, AAV3. 1/hu.6, AAV3. 1/hu.9, AAV3-9/rh.52, AAV3-1 1/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106. 1/hu.37, AAV1 14.3/hu.40, AAV127.2/hu.41, AAV127.5/hu.42, AAV128.3/hu.44, AAV130.4/hu.48, AAV145. 1/hu.53, AAV145.5/hu.54, AAV145.6/hu.55, AAV161. 10/hu.60, AAV161.6/hu.61, AAV33. 12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LKO1, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128. 1/hu.43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1 AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6. 1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1 AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLvl-1, AAV Clvl-10, AAV CLvl-2, AAV CLv-1, AAV CLv13, AAV CLv-13AAV CLvl-4, AAV Clvl-7, AAV Clvl-8, AAV Clvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-Kl, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1 AAV CLv-R2, AAV CLv-R3, AAV CLv-R4, AAV CLv-R5, AAV CLv-R6, AAV CLv-R7, AAV CLv-R8, AAV CLv-R9, AAV CSp-1 AAV CSp-10AAV CSp-11AAV CSp-2, AAV CSp-3, AAV CSp-4, AAV CSp-6, AAV CSp-7, AAV CSp-8, AAV CSp-8. 10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or any combination thereof.

Imaging Methods

Disclosed herein include methods of imaging a target site of a subject. In some embodiments, the method comprises: administering to the subject an effective amount of a viral vector composition disclosed herein; and applying ultrasound (US) to the target site of the subject to obtain a US image of the target site, optionally a nonlinear US image.

There are provided, in some embodiments, methods of imaging gene expression within a subject. In some embodiments, the method comprises: administering to the subject an effective amount of a viral vector composition disclosed herein; and applying ultrasound (US) to the target site of the subject to obtain a US image of gene expression at the target site, optionally a nonlinear US image.

Disclosed herein include methods of imaging gene expression within target cells. In some embodiments, the method comprises: transducing target cells with a viral vector composition disclosed herein; and applying ultrasound (US) to said target cells to obtain a US image of gene expression at the target site, optionally a nonlinear US image. The target cells can be target cells of a subject. The transducing step can comprise administering to the subject an effective amount of a viral vector composition disclosed herein.

There are provided, in some embodiments, methods of detecting a unique cell type and/or unique cell state within a subject. In some embodiments, the method comprises: administering to the subject an effective amount of a viral vector composition disclosed herein; and applying ultrasound (US) to the target site of the subject, thereby detecting the unique cell type and/or unique cell state within said subject.

Disclosed herein include methods of detecting a unique cell type and/or unique cell state. In some embodiments, the method comprises: transducing target cells with a viral vector composition disclosed herein; and applying ultrasound (US) to said target cells, thereby detecting the unique cell type and/or unique cell state. The target cells can be target cells of a subject. The transducing step can comprise administering to the subject an effective amount of a viral vector composition disclosed herein.

There are provided, in some embodiments, methods of detecting perturbation-induced changes in cell type and/or cell state. In some embodiments, the method comprises: transducing target cells with a viral vector composition disclosed herein; introducing one or more genetic, chemical, and/or physical perturbations to said target cells; and applying ultrasound (US) to said target cells, thereby detecting the perturbation-induced changes in cell type and/or cell state. The target cells can be target cells of a subject. The transducing step can comprise administering to the subject an effective amount of a viral vector composition disclosed herein.

The term “contrast enhanced imaging” or “imaging”, as used herein indicates a visualization of a target site performed with the aid of a contrast agent administered to the target site to improve the visibility of structures or fluids by devices process and techniques suitable to provide a visual representation of a target site. Accordingly a contrast agent is a substance that enhances the contrast of structures or fluids within the target site, producing a higher contrast image for evaluation.

The term “ultrasound imaging” or ultrasound scanning” or “sonography” as used herein indicate imaging performed with techniques based on the application of ultrasound. Ultrasound can refer to sound with frequencies higher than the audible limits of human beings, typically over 20 kHz. Ultrasound devices typically can range up to the gigahertz range of frequencies, with most medical ultrasound devices operating in the 1 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a skilled person.

Accordingly, the wording “ultrasound imaging” as used herein can refer to in particular to the use of high frequency sound waves, typically broadband waves in the megahertz range, to image structures in the body. The image can be up to 3D with ultrasound. In particular, ultrasound imaging typically involves the use of a small transducer (probe) transmitting high-frequency sound waves to a target site and collecting the sounds that bounce back from the target site to provide the collected sound to a computer using sound waves to create an image of the target site. Ultrasound imaging allows detection of the function of moving structures in real-time. Ultrasound imaging works on the principle that different structures/fluids in the target site will attenuate and return sound differently depending on their composition. A contrast agent sometimes used with ultrasound imaging are microbubbles created by an agitated saline solution, which works due to the drop in density at the interface between the gas in the bubbles and the surrounding fluid, which creates a strong ultrasound reflection. Ultrasound imaging can be performed with conventional ultrasound techniques and devices displaying 2D images as well as three-dimensional (3-D) ultrasound that formats the sound wave data into 3-D images. In addition to 3D ultrasound imaging, ultrasound imaging also encompasses Doppler ultrasound imaging, which uses the Doppler Effect to measure and visualize movement, such as blood flow rates. Types of Doppler imaging includes continuous wave Doppler, where a continuous sinusoidal wave is used; pulsed wave Doppler, which uses pulsed waves transmitted at a constant repetition frequency, and color flow imaging, which uses the phase shift between pulses to determine velocity information which is given a false color (such as red=flow towards viewer and blue=flow away from viewer) superimposed on a grey-scale anatomical image. Ultrasound imaging can use linear or non-linear propagation depending on the signal level. Harmonic and harmonic transient ultrasound response imaging can be used for increased axial resolution, as harmonic waves are generated from non-linear distortions of the acoustic signal as the ultrasound waves insonate tissues in the body. Other ultrasound techniques and devices suitable to image a target site using ultrasound would be understood by a skilled person.

The basic physics of sound waves enables ultrasound to visualize biological tissues with high spatial and temporal resolution. This capability has been enhanced by the development of acoustic biomolecules proteins with physical properties enabling them to scatter sound. The first acoustic biomolecules developed as contrast agents in ultrasound imaging, analogous to GFPs used in optical imaging, were based on a unique class of air-filled protein nanostructures called gas vesicles (GVs). The advancement of GVs has made it possible to use ultrasound to visualize the functions of cells deep inside tissues.

The term “ultrasound” can refer to sound with frequencies higher than the audible limits of human beings, typically over 20 kHz. Ultrasound devices typically can range up to the gigahertz range of frequencies, with most medical ultrasound devices operating in the 0.2 to 18 MHz range. The amplitude of the waves relates to the intensity of the ultrasound, which in turn relates to the pressure created by the ultrasound waves. Applying ultrasound can be accomplished, for example, by sending strong, short electrical pulses to a piezoelectric transducer directed at the target. Ultrasound can be applied as a continuous wave, or as wave pulses as will be understood by a person skilled in focused ultrasound. U.S. Patent Application Publication No. 2020/0237346 describes methods comprising the application of a step function increase in acoustic pressure during ultrasound imaging using gas vesicle contrast, along with capturing successive frames of ultrasound imaging and extracting time-series vectors for pixels of the frames, the content of which is hereby expressly incorporated by reference in its entirety. In some embodiments, the first, second, third, fourth, fifth, and/or sixth US pulse(s) each comprise a set of pulses.

Focused ultrasound (“FUS”) can refer to the technology that uses ultrasound energy to target specific areas of a subject, such as a specific area of a brain or body. FUS focuses acoustic waves by employing concave transducers that usually have a single geometric focus, or an array of ultrasound transducer elements which are actuated in a spatiotemporal pattern such as to produce one or more focal zones. At this focus or foci most of the power is delivered during sonication in order to induce mechanical effects, thermal effects, or both. The frequencies used for focused ultrasound are in the range of 200 KHz to 8000 KHz.

As used herein, the term “harmonic signal” or “harmonic frequency” can refer to a frequency in a periodic waveform that is an integer multiple of the frequency of the fundamental signal. In addition, this term encompasses sub-harmonic signals, which are signals with a frequency equal to an integral submultiple of the frequency of the fundamental signal. In ultrasound imaging, the transmitted pulse is typically centered around a fundamental frequency, and received signals may be processed to isolate signals centered around the fundamental frequency or one or more harmonic frequencies.

The term “fundamental signal” or “fundamental wave” can refer to the primary frequency of the transmitted ultrasound pulse. All GVs can backscatter ultrasound at the fundamental frequency, allowing their detection by ultrasound.

The term “non-linear signal” can refer to a signal that does not obey superposition and scaling properties, with regards to the input. The term “linear signal” can refer to a signal that does obey those properties. One example of non-linearity is the production of harmonic signals in response to ultrasound excitation at a certain fundamental frequency. Another example is a non-linear response to acoustic pressure. One embodiment of such a non-linearity is the acoustic collapse profile of GVs, in which there is a non-linear relationship between the applied pressure and the disappearance of subsequent ultrasound contrast from the GVs as they collapse. Another example of a non-linear signal that does not involve the destruction of GVs, is the increase in both fundamental and harmonic signals with increasing pressure of the transmitted imaging pulse, wherein certain GVs exhibit a super-linear relationship between these signals and the pulse pressure.

The term “applying ultrasound” shall be given its ordinary meaning, and shall also refer to sending ultrasound-range acoustic energy to a target. The sound energy produced by the piezoelectric transducer can be focused by beamforming, through transducer shape, lensing, or use of control pulses. The soundwave formed is transmitted to the body, then partially reflected or scattered by structures within a body; larger structures typically reflecting, and smaller structures typically scattering. The return sound energy reflected/scattered to the transducer vibrates the transducer and turns the return sound energy into electrical signals to be analyzed for imaging. The frequency and pressure of the input sound energy can be controlled and are selected based on the needs of the particular imaging/delivery task and, in some methods described herein, collapsing GVs (thereby inducing engineered cells herein to release payload molecules at a target site). To create images, particularly 2D and 3D imaging, scanning techniques can be used where the ultrasound energy is applied in lines or slices which are composited into an image.

In certain embodiments, the method includes applying a set of imaging pulses from an ultrasound transmitter to the target site, and receiving ultrasound signal at a receiver. In certain instances, the ultrasound signal detected by the receiver includes an ultrasound echo signal. Additional information of ultrasound systems and methods can be found in related publications as will be understood by a person skilled in the art.

Methods for performing ultrasound imaging are known in the art and can be employed in methods of the current disclosure. In certain aspects, an ultrasound transducer, which comprises piezoelectric elements, transmits an ultrasound imaging signal (or pulse) in the direction of the target site. Variations in the acoustic impedance (or echogenicity) along the path of the ultrasound imaging signal causes backscatter (or echo) of the imaging signal, which is received by the piezoelectric elements. The received echo signal is digitized into ultrasound data and displayed as an ultrasound image. Conventional ultrasound imaging systems comprise an array of ultrasonic transducer elements that are used to transmit an ultrasound beam, or a composite of ultrasonic imaging signals that form a scan line. The ultrasound beam is focused onto a target site by adjusting the relative phase and amplitudes of the imaging signals. The imaging signals are reflected back from the target site and received at the transducer elements. The voltages produced at the receiving transducer elements are summed so that the net signal is indicative of the ultrasound energy reflected from a single focal point in the subject. An ultrasound image is then composed of multiple image scan lines.

In some embodiments, imaging the target site is performed by applying or transmitting an imaging ultrasound signal from an ultrasound transmitter to the target site and receiving a set of ultrasound data at a receiver. The ultrasound data can be obtained using a standard ultrasound device, or can be obtained using an ultrasound device configured to specifically detect the contrast agent used. Obtaining the ultrasound data can include detecting the ultrasound signal with an ultrasound detector. In some embodiments, the imaging step further comprises analyzing the set of ultrasound data to produce an ultrasound image.

In certain embodiments, the ultrasound signal has a transmit frequency of at least 1 MHz, 5 MHz, 10 MHz, 20 MHz, 30 MHz, 40 MHz or 50 MHz. For example, an ultrasound data is obtained by applying to the target site an ultrasound signal at a transmit frequency from 4 to 11 MHz, or at a transmit frequency from 14 to 22 MHz. In some instances, the imaging frequency is selected so as to maximize the contrast generated by the administered contrast agent.

In the embodiments herein described, the collapsing ultrasound and imaging ultrasound are selected to have a collapsing pressure and an imaging pressure amplitude based on the acoustic collapse pressure profile (e.g., peak acoustic pressure) of the GVPS type used. In some instances, the ultrasound pressure, including the collapsing ultrasound pressure and the imaging ultrasound pressure can be referred to as the “peak positive pressure” of the ultrasound pulses. The term “peak positive pressure” can refer to the maximum pressure amplitude of the positive pulse of a pressure wave, typically in terms of the difference between the peak pressure and the ambient pressure at the location in the person or specimen that is being imaged.

In some embodiments, the administering step comprises: isolating target cells from the subject; transducing target cells with the viral vector composition disclosed herein; and administering to the subject an effective amount of the transduced cells. The method can comprise: administering an effective amount of a transactivator-binding compound to the subject. In some embodiments, the transactivator-binding compound comprises tetracycline, doxycycline or a derivative thereof. The administering can comprise systemic administration. The systemic administration can be intravenous, intramuscular, intraperitoneal, or intraarticular. Administering can comprise intracranial delivery, intrathecal administration, intracranial injection, aerosol delivery, nasal delivery, vaginal delivery, direct injection to any tissue in the body, intraventricular delivery, intraocular delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The period of time between the administering and applying can be about 21 days, about 14 days, about 7 days, about 3 days, about 48 hours, about 44 hours, about 40 hours, about 35 hours, about 30 hours, about 25 hours, 20 hours, 15 hours, 10 hours, about 8 hours, about 7 hours, about 6 hours, about 5 hours, about 4 hours, about 3 hours, about 2 hours, about 1 hour, about 30 minutes, about 15 minutes, about 10 minutes, or about 5 minutes.

The target cells can be in vitro, in vivo, and/or ex vivo. The target cells can be tissue culture cells. The target cell(s) can comprise an immortalized cells or primary cells. The target cell can comprise a eukaryotic cell. The eukaryotic cell can comprise or be an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The target cells can be situated within a target site of a subject, and applying US can comprise applying US to the target site to obtain a US image of the target site. In some embodiments, the target cells in a unique cell type and/or a unique cell state express GVs, and wherein said GVs produce nonlinear ultrasound contrast. In some embodiments, applying US causes the production of unique cell type-dependent and/or unique cell state-dependent nonlinear ultrasound contrast. Detecting a unique cell type and/or unique cell state can comprise detecting an at least about 5 dB enhancement in nonlinear ultrasound contrast. Applying US can comprise nonlinear US imaging. Applying US can comprise applying one or more US pulse(s) over a duration of time. The duration of time can be about 48 hours, about 44 hours, about 40 hours, about 35 hours, about 30 hours, about 25 hours, 20 hours, 15 hours, 10 hours, about 8 hours, about 8 hours, 8 hours, about 7 hours, about 6 hours, about 5 hours, about 4 hours, about 3 hours, about 2 hours, about 1 hour, about 30 minutes, about 15 minutes, about 10 minutes, or about 5 minutes. In some embodiments, the one or more US pulse(s) each have a pulse duration of about 1 hour, about 30 minutes, about 15 minutes, about 10 minutes, about 5 minutes, about 1 minute, about 1 second, or about 1 millisecond.

Applying one or more US pulse(s) can comprise applying one or more focused US pulse(s). Applying one or more US pulse(s) can comprise applying US at a frequency of 100 kHz to 100 MHz. Applying one or more US pulse(s) can comprise applying ultrasound at a frequency of 0.2 to 1.5 mHz. Applying one or more US pulse(s) can comprise applying ultrasound having a mechanical index in a range between 0.2 and 0.6. The one or more US pulse(s) can comprise a peak pressure of about 40 kPa to about 800 kPa. The one or more US pulse(s) can comprise a peak pressure of about 70 kPa to about 150 kPa, and/or about 440 kPa to about 605 kPa.

The one or more US pulse(s) can comprise a pressure value that is: the selectable buckling pressure; the first optimum buckling pressure; and/or equal to or less than the first initial collapse pressure. In some embodiments, the one or more US pulse(s) induces collapse of GVs, and wherein the one or more US pulse(s) comprise a pressure value that is the first selectable collapse pressure. The nonlinear ultrasound imaging can comprise cross-amplitude modulation (x-AM) ultrasound imaging or parabolic amplitude modulation (pAM) ultrasound imaging. The nonlinear ultrasound imaging can comprise differential nonlinear ultrasound imaging. Differential nonlinear ultrasound imaging can comprise imaging of the second and/or higher harmonics with the first harmonic signal subtracted out. The nonlinear ultrasound imaging can comprise cross-phase modulation imaging and/or harmonic imaging. The nonlinear ultrasound imaging can comprise providing amplitude modulation (AM) ultrasound pulse sequences.

In some embodiments, the nonlinear ultrasound imaging comprises: pairs of cross-propagating plane waves to elicit nonlinear scattering from buckling GVs at the wave intersection; subtracting the signal generated by transmitting each wave on its own; and quantifying the resulting contrast. In some embodiments, the signals generated by transmitting each wave on its own has linear characteristics and/or lower nonlinear characteristics than the combined transmission of both plane waves produced at their intersection.

In some embodiments, the nonlinear ultrasound imaging comprises: a peak positive pressure of two single tilted plane waves exciting the GV in a linear scattering regime; a doubled X-wave intersection amplitude exciting the GV in a nonlinear scattering regime; summing the echoes from the two single tilted plane-wave transmissions to generate a sum; and subtracting the sum from the echoes of the X-wave transmissions to derive nonzero differential GV signals.

Applying US can comprise detecting scattering of the one or more US pulse(s) by gas vesicles (e.g., nonlinear scattering of the US by buckling gas vesicles). In some embodiments, detecting scattering comprises: detecting backscattered echoes of two half-amplitude transmissions at applied pressures below the buckling threshold of the GV; and detecting backscattered echoes of a third full-amplitude transmission at pressures above the buckling threshold of the gas vesicles. In some embodiments, said two half-amplitude transmissions trigger largely linear scattering. In some embodiments, said third full-amplitude transmission triggers harmonic and nonlinear scattering. The method can comprise subtracting the backscattered echoes of the two half-amplitude transmissions from the backscattered echoes of the third full-amplitude transmission.

In some embodiments, the method comprises: single-cell imaging; and/or imaging a large volume in deep tissue. The method can comprise US imaging with a spatiotemporal resolution of less than about 100 μm (e.g., 100 μm, 10 μm, 1 μm, 0.1 μm, 0.01 μm, 0.001 μm or a range between any two of these values) and less than about 1 ms (e.g., 1 ms, 0.1 ms, 0.01 ms, 0.001 ms, or a range between any two of these values). In some embodiments, the target site comprises: a volume larger than about 1 mm³ (e.g., 1 mm³, 10 mm³, 100 mm³, 1000 mm³, 10000 mm³, 100000 mm³, or a range between any two of these values); a depth deeper than about 1 mm; (e.g., 1 mm, 10 mm, 100 mm, 1000 mm, 10000 mm, 100000 mm, or a range between any two of these values); a depth and/or a volume inaccessible via optical imaging and/or fiber photometry; and/or the entire brain or a portion thereof.

The viral vector composition can be capable of expressing gas vesicle(s) having an acoustic collapse pressure threshold, and wherein applying ultrasound comprises: applying ultrasound to the target site at a peak positive pressure less than the acoustic collapse pressure threshold; increasing peak positive pressure (PPP) to above the selective acoustic collapse pressure value as a step function; and imaging the target site in successive frames during the increasing; and extracting a time-series vector for each of at least one pixel of the successive frames. In some embodiments, the method comprises: performing a signal separation algorithm on the time-series vectors using at least one template vector. In some embodiments, the signal separation algorithm includes template projection and/or template unmixing. In some embodiments, the at least one template vector includes linear scatterers, noise, gas vesicles, or a combination thereof. The successive frames can comprise a frame prior to GVs collapse, a frame during GVs collapse, and a frame after GVs collapse. In some embodiments, the increasing includes increasing the PPP to a hiBURST regime, optionally the PPP in hiBURST regime is 4.3 MPa or higher. In some embodiments, the increasing includes increasing the PPP to a loBURST regime, optionally the PPP in loBURST regime is no higher than 3.7 MPa.

In some embodiments, multiplexed imaging methods are provided. The term “multiplex” can refer to the presence of two or more distinct GVPS types, each of which exhibits an acoustic collapse pressure profile distinct from one another, in the transduced cells. The two or more distinct GVPSs can be derived from different organisms or variants of GVPSs from the same or different organisms (e.g., archaea). In multiplexing methods herein described, both the collapsing pressure of the collapsing ultrasound and the imaging pressure of the imaging ultrasound are selected based on the acoustic collapse pressure profiles of the GVPS types (e.g., peak acoustic pressure) to selectively collapse one GVPS type over the other GVPS types. The term “selectively collapse” can refer to collapsing at least a portion of one GVPS type in a greater amount that any other GVPS type in a mixture containing a plurality of GVPS types.

The subject can be a mammal. In some embodiments, the subject is not anesthetized. The target site can comprise a site of disease or disorder or can be proximate to a site of a disease or disorder. The target site can comprise a tissue. The tissue can be inflamed tissue and/or infected tissue. The tissue can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. In some embodiments, the tissue comprises: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue. The nonlinear ultrasound imaging can comprise transcranial imaging, and the target site can comprise one or more target brain region(s). The target brain region(s) can comprise the Lateral parabrachial nucleus, brainstem, Medulla oblongata, Medullary pyramids, Olivary body, Inferior olivary nucleus, Rostral ventrolateral medulla, Respiratory center, Dorsal respiratory group, Ventral respiratory group, Pre-Botzinger complex, Botzinger complex, Paramedian reticular nucleus, Cuneate nucleus, Gracile nucleus, Intercalated nucleus, Area postrema, Medullary cranial nerve nuclei, Inferior salivatory nucleus, Nucleus ambiguus, Dorsal nucleus of vagus nerve, Hypoglossal nucleus, Solitary nucleus, Pons, Pontine nuclei, Pontine cranial nerve nuclei, chief or pontine nucleus of the trigeminal nerve sensory nucleus (V), Motor nucleus for the trigeminal nerve (V), Abducens nucleus (VI), Facial nerve nucleus (VII), vestibulocochlear nuclei (vestibular nuclei and cochlear nuclei) (VIII), Superior salivatory nucleus, Pontine tegmentum, Respiratory centers, Pneumotaxic center, Apneustic center, Pontine micturition center (Barrington's nucleus), Locus coeruleus, Pedunculopontine nucleus, Laterodorsal tegmental nucleus, Tegmental pontine reticular nucleus, Superior olivary complex, Paramedian pontine reticular formation, Cerebellar peduncles, Superior cerebellar peduncle, Middle cerebellar peduncle, Inferior cerebellar peduncle, Cerebellum, Cerebellar vermis, Cerebellar hemispheres, Anterior lobe, Posterior lobe, Flocculonodular lobe, Cerebellar nuclei, Fastigial nucleus, Interposed nucleus, Globose nucleus, Emboliform nucleus, Dentate nucleus, Tectum, Corpora quadrigemina, inferior colliculi, superior colliculi, Pretectum, Tegmentum, Periaqueductal gray, Parabrachial area, Medial parabrachial nucleus, Subparabrachial nucleus (Kolliker-Fuse nucleus), Rostral interstitial nucleus of medial longitudinal fasciculus, Midbrain reticular formation, Dorsal raphe nucleus, Red nucleus, Ventral tegmental area, Substantia nigra, Pars compacta, Pars reticulata, Interpeduncular nucleus, Cerebral peduncle, Crus cerebri, Mesencephalic cranial nerve nuclei, Oculomotor nucleus (III), Trochlear nucleus (IV), Mesencephalic duct (cerebral aqueduct, aqueduct of Sylvius), Pineal body, Habenular nucleim Stria medullares, Taenia thalami, Subcommissural organ, Thalamus, Anterior nuclear group, Anteroventral nucleus (aka ventral anterior nucleus), Anterodorsal nucleus, Anteromedial nucleus, Medial nuclear group, Medial dorsal nucleus, Midline nuclear group, Paratenial nucleus, Reuniens nucleus, Rhomboidal nucleus, Intralaminar nuclear group, Centromedial nucleus, Parafascicular nucleus, Paracentral nucleus, Central lateral nucleus, Central medial nucleus, Lateral nuclear group, Lateral dorsal nucleus, Lateral posterior nucleus, Pulvinar, Ventral nuclear group, Ventral anterior nucleus, Ventral lateral nucleus, Ventral posterior nucleus, Ventral posterior lateral nucleus, Ventral posterior medial nucleus, Metathalamus, Medial geniculate body, Lateral geniculate body, Thalamic reticular nucleus, Hypothalamus, limbic system, HPA axis, preoptic area, Medial preoptic nucleus, Suprachiasmatic nucleus, Paraventricular nucleus, Supraoptic nucleusm Anterior hypothalamic nucleus, Lateral preoptic nucleus, median preoptic nucleus, periventricular preoptic nucleus, Tuberal, Dorsomedial hypothalamic nucleus, Ventromedial nucleus, Arcuate nucleus, Lateral area, Tuberal part of Lateral nucleus, Lateral tuberal nuclei, Mammillary nuclei, Posterior nucleus, Lateral area, Optic chiasm, Subfornical organ, Periventricular nucleus, Pituitary stalk, Tuber cinereum, Tuberal nucleus, Tuberomammillary nucleus, Tuberal region, Mammillary bodies, Mammillary nucleus, Subthalamus, Subthalamic nucleus, Zona incerta, Pituitary gland, neurohypophysis, Pars intermedia, adenohypophysis, cerebral hemispheres, Corona radiata, Internal capsule, External capsule, Extreme capsule, Arcuate fasciculus, Uncinate fasciculus, Perforant Path, Hippocampus, Dentate gyms, Cornu ammonis, Cornu ammonis area 1, Cornu ammonis area 2, Cornu ammonis area 3, Cornu ammonis area 4, Amygdala, Central nucleus, Medial nucleus (accessory olfactory system), Cortical and basomedial nuclei, Lateral and basolateral nuclei, extended amygdala, Stria terminalis, Bed nucleus of the stria terminalis, Claustrum, Basal ganglia, Striatum, Dorsal striatum (aka neostriatum), Putamen, Caudate nucleus, Ventral striatum, Striatum, Nucleus accumbens, Olfactory tubercle, Globus pallidus, Subthalamic nucleus, Basal forebrain, Anterior perforated substance, Substantia innominata, Nucleus basalis, Diagonal band of Broca, Septal nuclei, Medial septal nuclei, Lamina terminalis, Vascular organ of lamina terminalis, Olfactory bulb, Piriform cortex, Anterior olfactory nucleus, Olfactory tract, Anterior commissure, Uncus, Cerebral cortex, Frontal lobe, Frontal cortex, Primary motor cortex, Supplementary motor cortex, Premotor cortex, Prefrontal cortex, frontopolar cortex, Orbitofrontal cortex, Dorsolateral prefrontal cortex, dorsomedial prefrontal cortex, ventrolateral prefrontal cortex, Superior frontal gyms, Middle frontal gyms, Inferior frontal gyms, Brodmann areas (4, 6, 8, 9, 10, 11, 12, 24, 25, 32, 33, 44, 45, 46, and/or 47), Parietal lobe, Parietal cortex, Primary somatosensory cortex (S1), Secondary somatosensory cortex (S2), Posterior parietal cortex, posteentral gyms, precuneus, Brodmann areas (1, 2, 3 (Primary somesthetic area), 5, 7, 23, 26, 29, 31, 39, and/or 40), Occipital lobe, Primary visual cortex (V1), V2, V3, V4, V5/MT, Lateral occipital gyms, Cuneus, Brodmann areas (17 (V1, primary visual cortex), 18, and/or 19), temporal lobe, Primary auditory cortex (A1), secondary auditory cortex (A2), Inferior temporal cortex, Posterior inferior temporal cortex, Superior temporal gyms, Middle temporal gyms, Inferior temporal gyms, Entorhinal Cortex, Perirhinal Cortex, Parahippocampal gyms, Fusiform gyms, Brodmann areas (9, 20, 21, 22, 27, 34, 35, 36, 37, 38, 41, and/or 42), Medial superior temporal area (MST), insular cortex, cingulate cortex, Anterior cingulate, Posterior cingulate, dorsal cingulate, Retrosplenial cortex, Indusium griseum, Subgenual area 25, Brodmann areas (23, 24; 26, 29, 30 (retrosplenial areas), 31, and/or 32), cranial nerves (Olfactory (I), Optic (II), Oculomotor (III), Trochlear (IV), Trigeminal (V), Abducens (VI), Facial (VII), Vestibulocochlear (VIII), Glossopharyngeal (IX), Vagus (X), Accessory (XI), Hypoglossal (XII)), or any combination thereof. The target brain region can comprise neural pathways, Superior longitudinal fasciculus, Arcuate fasciculus, Thalamocortical radiations, Cerebral peduncle, Corpus callosum, Posterior commissure, Pyramidal or corticospinal tract, Medial longitudinal fasciculus, dopamine system, Mesocortical pathway, Mesolimbic pathway, Nigrostriatal pathway, Tuberoinfundibular pathway, serotonin system, Norepinephrine Pathways, Posterior column-medial lemniscus pathway, Spinothalamic tract, Lateral spinothalamic tract, Anterior spinothalamic tract, or any combination thereof.

The target site can comprise one or more target brain region(s). The target brain region(s) can comprise one or more target brain cell(s). The target cells can comprise one or more target brain cell(s). The unique cell type and/or unique cell state can be a target brain cell activity with respect to a neural circuit, a behavior, a physiological function and/or a condition associated with a target brain cell activity with respect to a neural circuit of the individual. In some embodiments, the target brain cell activity indicates a series of biological and biochemical reactions resulting in a direct or indirect effect on the synapses of the neural circuit and related passage of the electrochemical signals. Exemplary target brain cell activity in the sense of the disclosure comprise action potential, intrinsic electroresponsive properties like intrinsic transmembrane voltage oscillatory patterns, and production and/or release of chemicals such as neurotransmitters, gliotransmitters, and additional chemicals identifiable by a skilled person. A target cell activity with respect to a neural circuit can also be associated with a behavior and/or physiological function of the individual. The wording “associated to” as used herein with reference to two items indicates a relation between the two items such that the occurrence of a first item is accompanied by the occurrence of the second item, which includes but is not limited to a cause-effect relation and sign/symptoms-disease relation. Accordingly, disclosed herein include methods to detect a target brain cell activity with respect to a neural circuit.

Also disclosed herein are pharmaceutical compositions comprising one or more of the viral vector compositions disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). In some embodiments, the physiologically acceptable carrier is an aqueous pH buffered solution

Titers of the viral vectors provided herein to be administered will vary depending, for example, on the particular viral vector(s), the mode of administration, the subject, and the cell type(s) being targeted, and can be determined by methods standard in the art. As will be readily apparent to one skilled in the art, the useful in vivo dosage of the viral vector(s) to be administered and the particular mode of administration will vary depending upon the age, weight, and animal species treated, the particular viral vector(s) that is used, and the specific use for which the viral vector(s) is employed. The determination of effective dosage levels, that is the dosage levels necessary to achieve the desired result, can be accomplished by one skilled in the art using routine pharmacological methods.

In some embodiments, pharmaceutical compositions in accordance with the present disclosure is administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the effect. It will be understood that the above dosing concentrations may be converted to vg or viral genomes per kg or into total viral genomes administered by one of skill in the art.

In some embodiments, a dose of the pharmaceutical composition comprises a concentration of infectious particles of at least or about 10⁷, 10⁸, 10⁹, 10¹⁰, 10¹¹, 10¹², 10¹3, 10¹⁴, 10¹⁵, 10¹⁶, or 10¹⁷. In some cases the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, 2×10¹⁷, or a range between any two of these values. In some cases the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹, 4×10¹², 4×10¹³, 4×10¹⁴, 4×10¹⁵, 4×10¹⁶, 4×10¹⁷, or a range between any two of these values. In some cases the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10⁶, 7×10¹⁷, or a range between any two of these values. In some cases the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

The viral vector(s) provided herein can comprise tropism for a target tissue or a target cell. The target tissue or the target cell can comprise a tissue or a cell of a target brain region(s). The target cell can be a neuronal cell, a neural stem cell, an astrocytes, or a tumor cell. The target cell can be located in a brain or spinal cord. The target cell can comprise an antigen-presenting cell, a dendritic cell, a macrophage, a neural cell, a brain cell, an astrocyte, a microglial cell, and a neuron. In some embodiments, the target cell is an endothelial cell.

### EXAMPLES

Some aspects of the embodiments discussed above are disclosed in further detail in the following examples, which are not in any way intended to limit the scope of the present disclosure.

### Example 1

### Viral Delivery of Gas Vesicle Genes

Summary

To enable modular and robust delivery of GV genes, viral vector architectures were developed that successfully co-express more than 9 genes to produce strong acoustic contrast in a variety of cell types, including neuronal and immune cells. GV expression was then linked to cellular function by expressing GVs under neuronal activity-dependent promoters. The in vivo application of this technology was demonstrated by injecting the disclosed engineered viral vectors deep inside the mouse brain and imaging in situ GV expression at the site of injection. The disclosed viral GV delivery platform serves as a critical resource to greatly expand the in vivo imaging applications of acoustic reporter genes.

One of the most challenging aspects of mammalian GVs expression is ensuring robust and strong co-expression of 8-14 genes. The small GV gene cluster derived from cyanobacterium Anabaena flos-aquae that has been successfully cloned and expressed in mammalian cells with transfection-based methods was employed herein. This gene cluster consists of a structural gene, gvpA, and 7 chaperone GV proteins: gvpN, J, K, F, G, W, and V. Occupying 4.5 thousand base pair nucleotides in its coding sequence, this set of genes is suitable for packaging inside a lentivirus, an enveloped single-stranded RNA retrovirus. Herein, lentiviral transgene vectors expressing the Anabaena gene cluster under an inducible promoter were designed and it was shown that a variety of cell types transduced with the disclosed engineered viruses produce strong inducible nonlinear acoustic contrast.

To demonstrate the ability to link GV expression to cellular function, GVs were expressed under a variety of activity dependent promoters that are regulated by immediate early genes (IEGs). These promoters are widely used in neuroscience research to study fear conditioning, memory formation, and learning, among others. It is shown therein that inducing activity in IEG-driven GV expressing cells results in significant acoustic contrast compared to their resting state.

To demonstrate the in vivo potential of the disclosed viral delivery platforms to cause GV expression in endogenous primary cells, intracranial injection of GV-encoding viruses deep inside the brain of 6-8 week old mice was performed. GV expression around the site of injection was confirmed by nonlinear imaging of the brain. This is the first demonstration of US imaging of in situ gene expression in intact brain.

Results

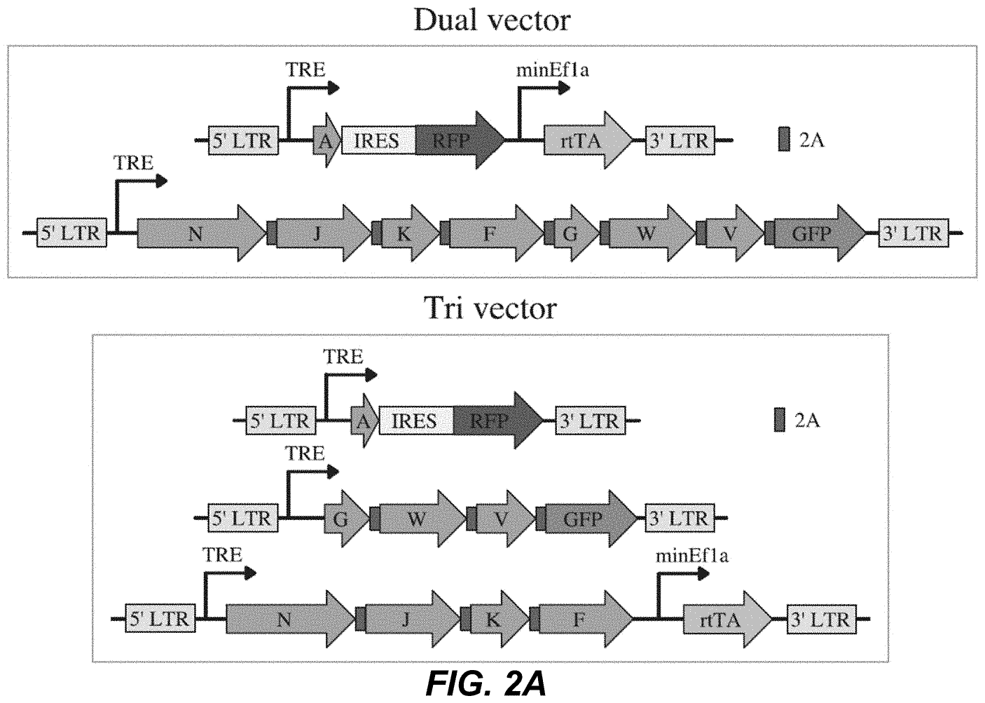

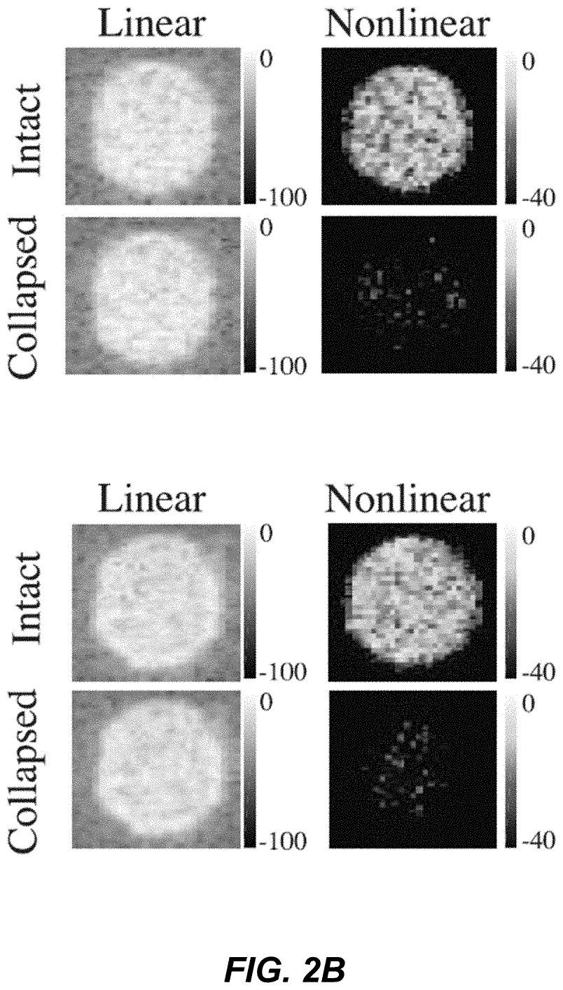

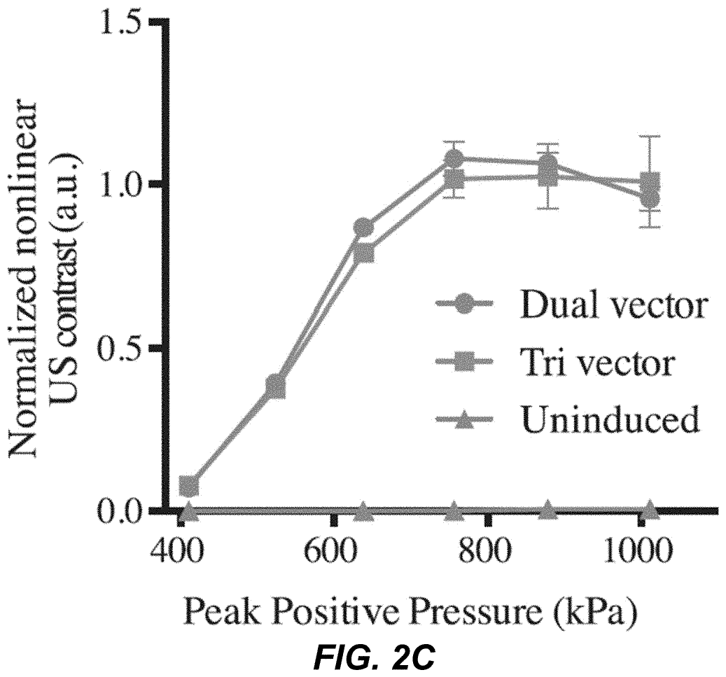

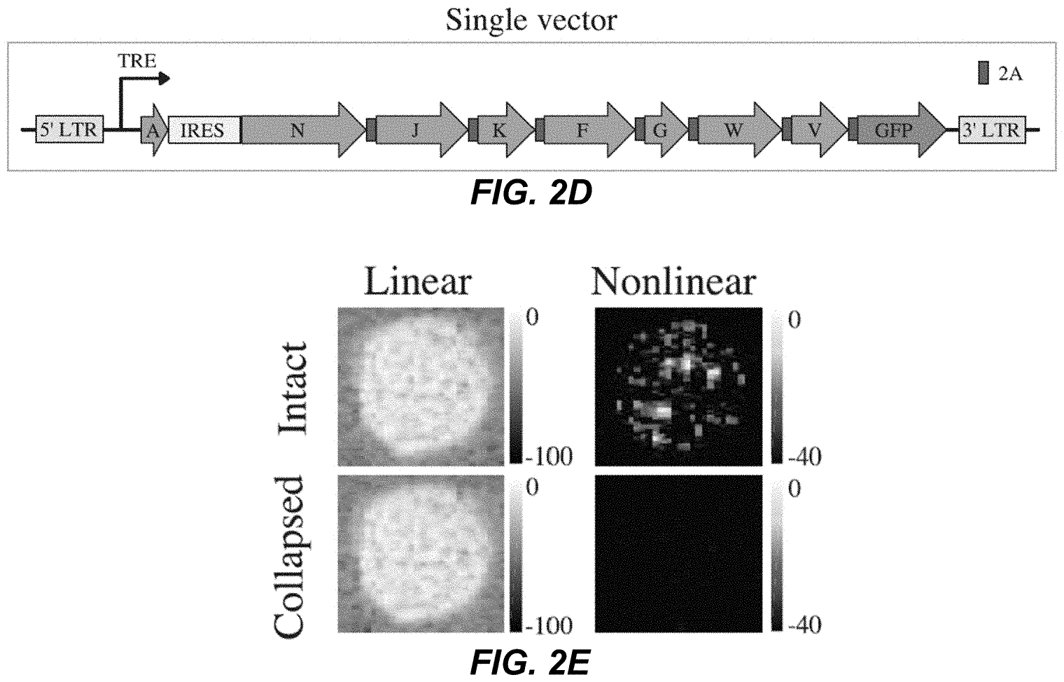

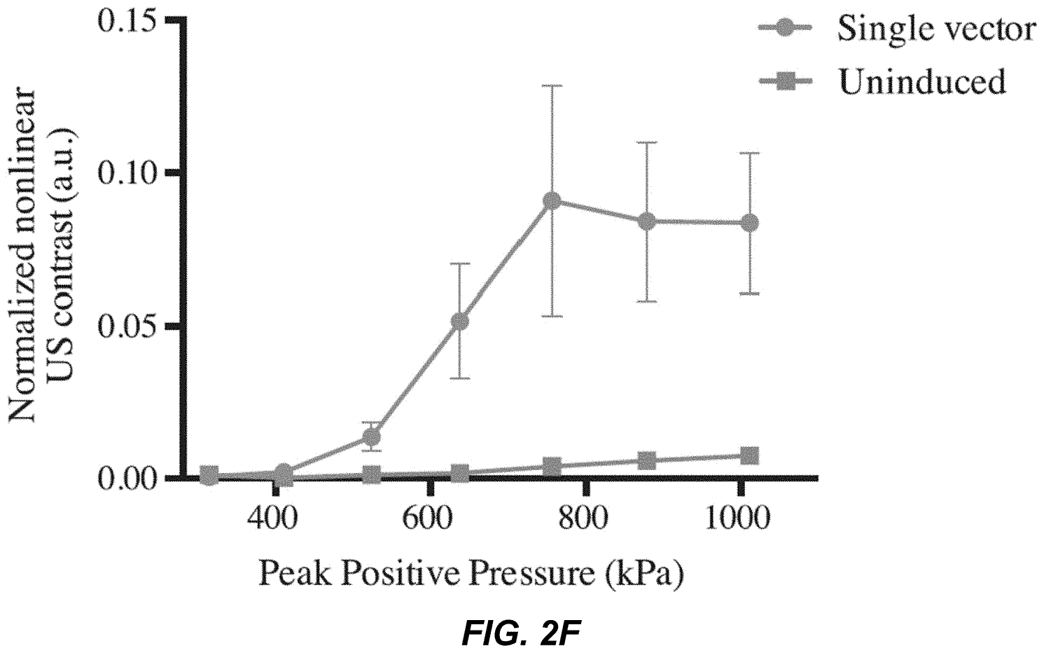

Various viral architectures were designed that require co-transduction of two or three viruses to express GVs inside a cell (FIG. 2A). In all designs, 2A linker peptides were used for poly-clonal expression of GV genes under a single promoter, with the exception of the gvpA protein which does not produce GVs in the presence of a 2A linker on either terminus. The 2A linker peptides are co-translational self-cleaving elements, which allow multiple proteins to be processively translated from a single mRNA in eukaryotic cells. In some embodiments of the constructs disclosed herein P2A, T2A and E2A variants of these linker peptides were employed. Additionally, in some embodiments, the disclosed design takes advantage of inducibility and strong transcription rate of the Tet-ON 3G system. In presence of doxycycline, a tetracycline derivative, the rtTA transactivator binds to the TRE promoter and turns on transcription. Vectors were designed containing all the necessary GV genes, the rtTA transgene, and fluorescent proteins, in either a dual-viral vector system or a tri-viral vector system and it was shown that co-transduction of these vectors produces strong nonlinear acoustic contrast (xAM imaging) in HEK293T cells (FIGS. 2B-2C). The nonlinear signal increased with acoustic pressure until high pressure results in collapse of GVs. The full collapse of GVs confirmed the specificity of the signal.

With 4.5 thousand base pair nucleotides in its coding sequence, the Anabaena gene cluster is small enough to be packaged into a single lentivirus. A vector was designed encoding all 8 GV genes and an internal ribosome entry site (IRES) was employed to express GvpA and the chaperones under a single TRE promoter. HEK-TetON cells transduced with this virus showed nonlinear signal in intact cells (FIGS. 2D-2F). The detected signal was weaker than that of the dual or tri-vector combinations. The lower expression was expected as previous studies have shown a negative correlation between lentivirus titer and its packaging size.

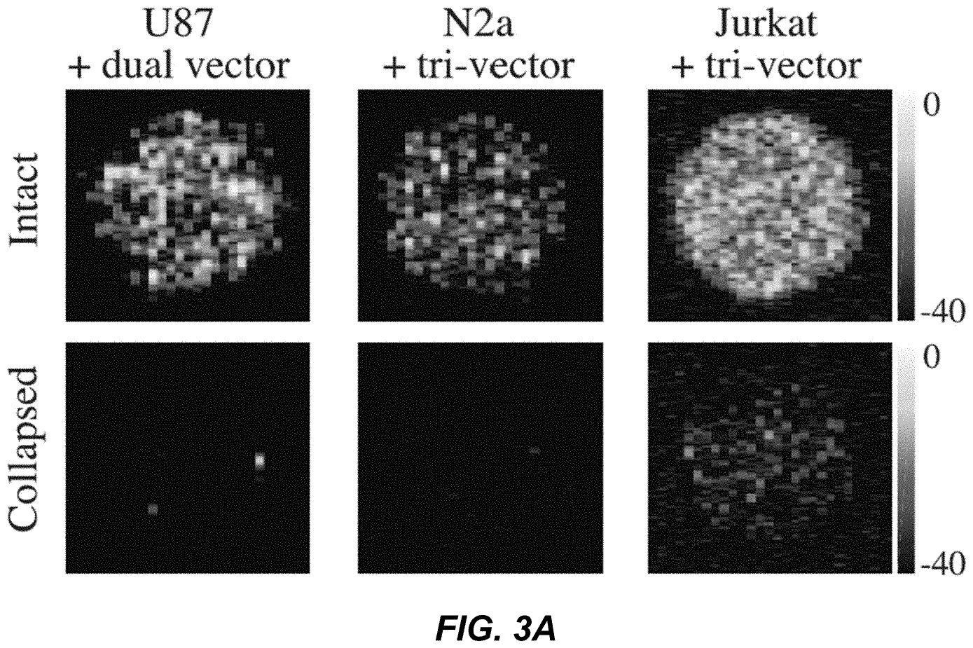

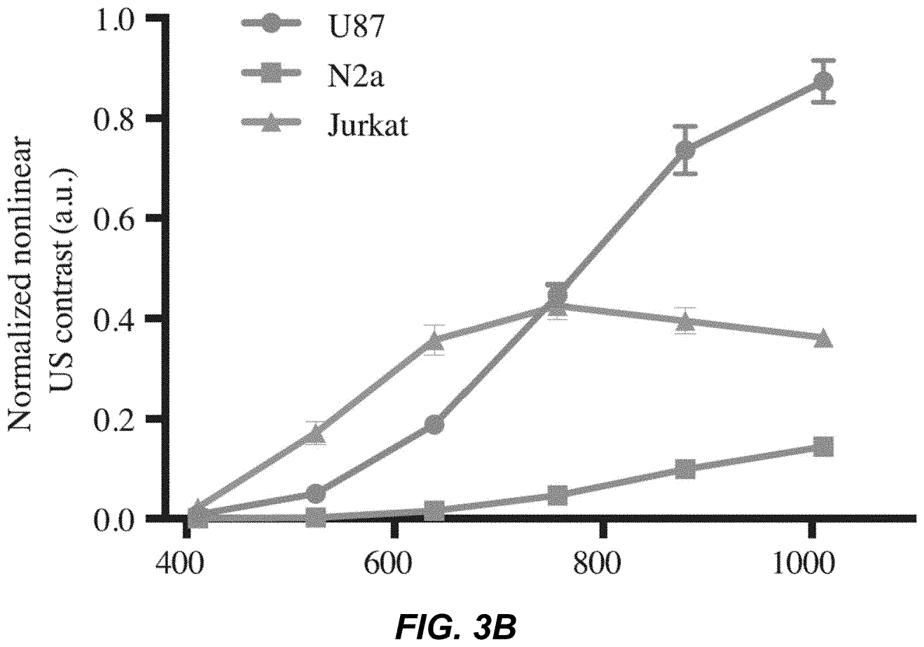

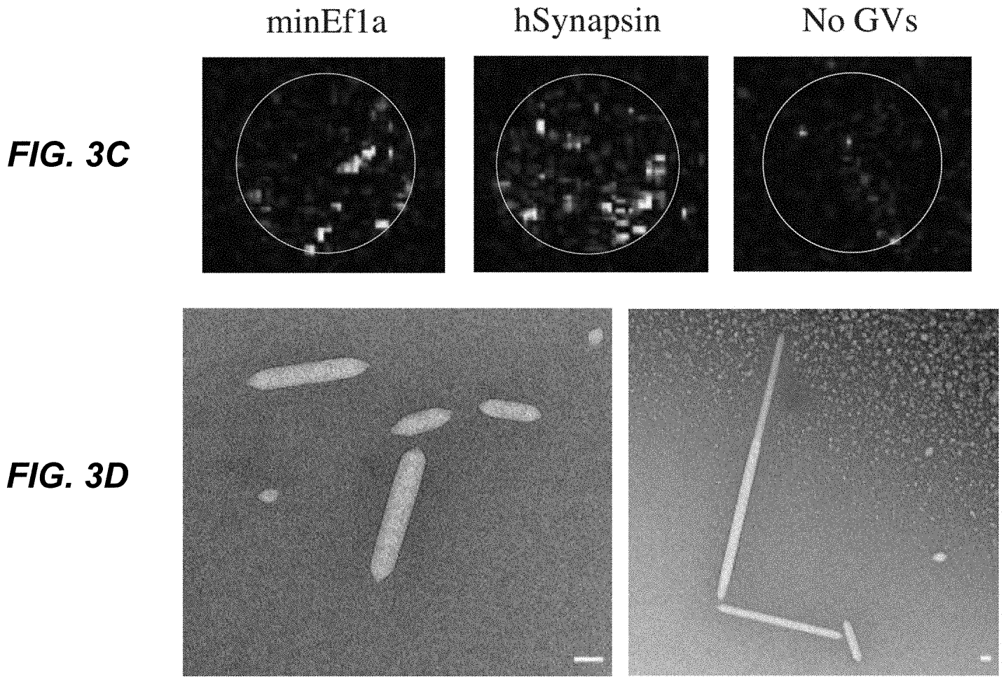

The disclosed viral platform transduces GVs in a cell-type independent manner, as demonstrated by successful transduction of neuronal cell lines U87 and N2a, and immortalized immune cell line Jurkats (FIGS. 3A-3B). Moreover, there was successful expression of GVs in mouse embryonic cortical neurons under minEf1a, a ubiquitous promoter, and hSynapsin, a neuron specific promoter (FIG. 3C). TEM grids of GVs floating in the lysate of cortical neurons confirmed that the nonlinear US contrast is due to collapse of GVs (BURST imaging) (FIG. 3D).

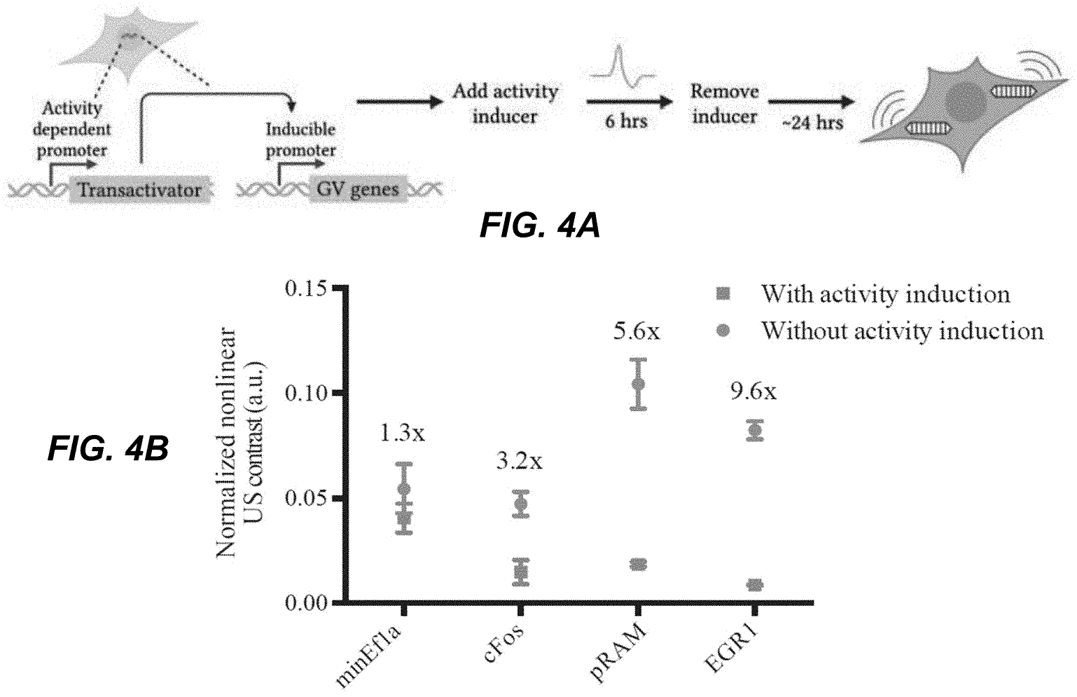

To link GV expression to cellular function, GVs were expressed under a variety of activity dependent promoters in HEK293T cells. Specifically, the constitutive promoter driving rtTA were swapped with cFos, pRAM (based on the minimal cFos promoter), and EGR1. These IEG regulated promoters become activated in response to increased intracellular activity. Activity was induced by adding 1.3 μM ionomycin, increasing intracellular level of calcium, and removing it after 6 hours. In 24 hours, the activity induced cells showed large nonlinear contrast, while the resting cells showed very little contrast (FIGS. 4A-4B). The large fold change in signal between the two conditions was not apparent in cells expressing GVs under a constitutive promoter. These experiments demonstrate the potential use of IEG-driven GV expression in noninvasive imaging of neural activity.

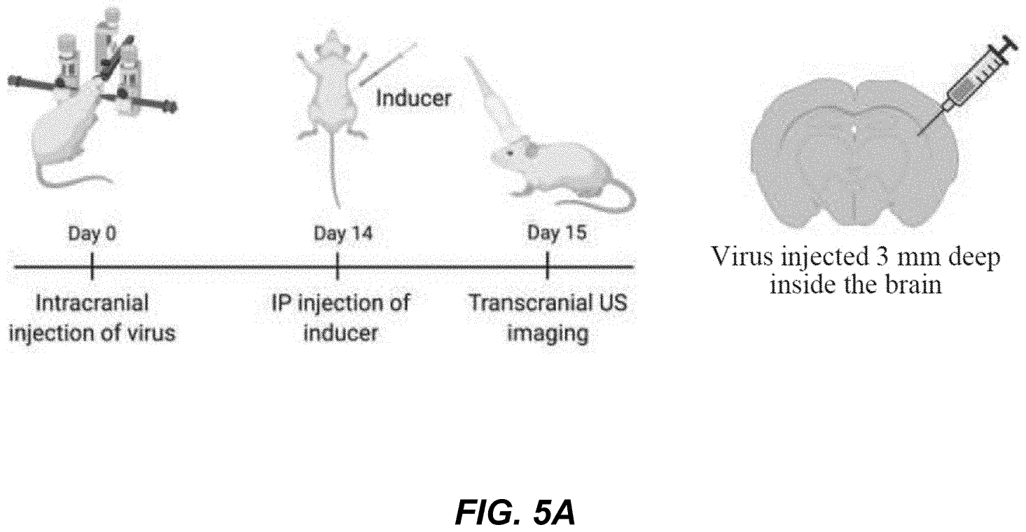

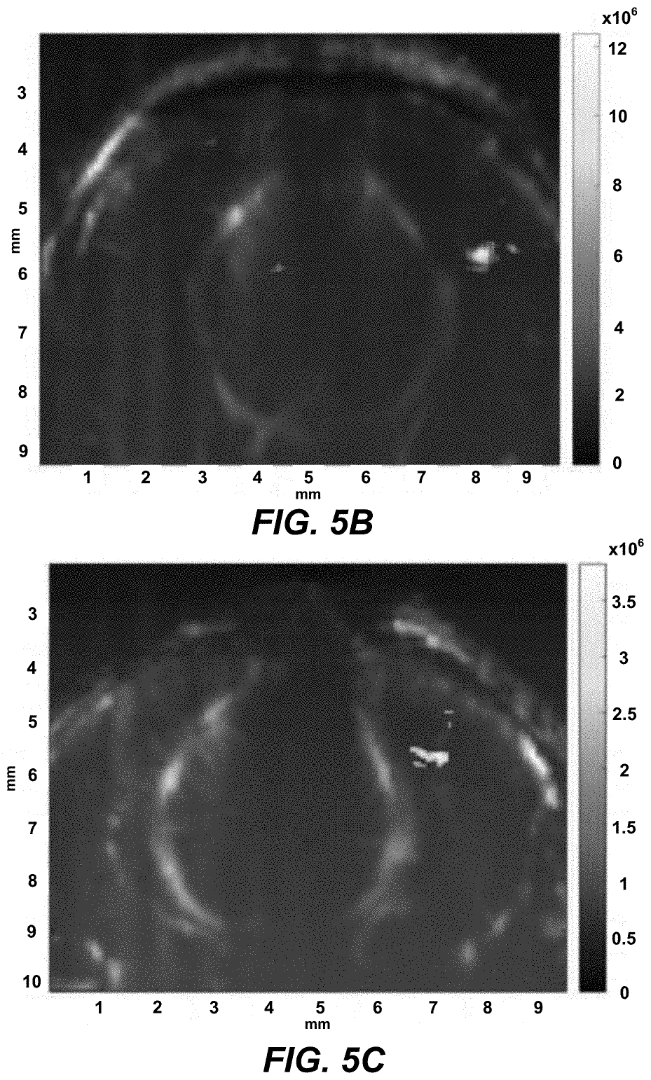

Next, it was determined whether GV-encoding lentiviruses enable transcranial imaging of in situ gene expression in the brain. The tri-viral vector lentiviruses encoding GVs under minEf1a were injected 3 mm deep inside the brain of 6-8 week old mice. 14 days post injection, GV expression was induced via intraperitoneal injection (IP) of doxycycline to activate the rtTA transactivator (FIG. 5A). GV expression around the site of injection was confirmed by nonlinear imaging (parabolic AM) of the brain and overlaid on functional ultrasound (fUS) images of the brain to show brain anatomy (FIGS. 5B-5C).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “of” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A viral vector composition, comprising:
a single viral vector comprising one or more first promoters operably connected to one or more gas vesicle (GV) polynucleotides comprising:
one or more gas vesicle assembly (GVA) gene(s) encoding one or more GVA protein(s), and
one or more gas vesicle structural (GVS) gene(s) encoding one or more GVS protein(s),
wherein the one or more GVA protein(s) and the one or more GVS protein(s) are capable of forming gas vesicles (GVs) upon expression in a cell.

2. The viral vector composition of claim 1, wherein the single viral vector comprises:
a context-dependent promoter operably linked to a transactivator polynucleotide comprising a transactivator gene,
wherein the context-dependent promoter is capable of inducing transcription of the transactivator gene to generate a transactivator transcript,
wherein the transactivator transcript is capable of being translated to generate a transactivator,
wherein the activity of the context-dependent promoter and/or the degree of expression of the transactivator is associated with the presence and/or amount a unique cell type and/or a unique cell state,
wherein, in the presence of the transactivator and a transactivator-binding compound, the first promoter is capable of inducing transcription of the one or more GV polynucleotides to generate GV transcript(s), and
wherein the GV transcript(s) are capable of being translated to generate GVA protein(s) and/or GVS protein(s).

3. The viral vector composition of claim 2, wherein the first promoter comprises one or more copies of a transactivator recognition sequence the transactivator is capable of binding to induce transcription, wherein the transactivator is incapable of binding the transactivator recognition sequence in the absence of the transactivator-binding compound, and wherein the one or more copies of a transactivator recognition sequence comprise one or more copies of a tet operator (TetO).

4. The viral vector composition of claim 2,
wherein the one or more copies of a transactivator recognition sequence comprise one or more copies of a tet operator (TetO);
wherein the first promoter comprises a tetracycline response element (TRE), and
wherein the TRE comprises one or more copies of a tet operator (TetO);
wherein the transactivator comprises reverse tetracycline-controlled transactivator (rtTA);
wherein the transactivator comprises tetracycline-controlled transactivator (tTA); and/or
wherein the transactivator-binding compound comprises tetracycline, doxycycline or a derivative thereof.

5. The viral vector composition of claim 2, wherein the degree of expression of the transactivator is positively correlated with the presence and/or amount a unique cell type and/or a unique cell state

6. The viral vector composition of claim 2, wherein the context-dependent promoter is an Immediate Early Gene (IEG) regulated promoter, cFos, pRAM, EGR1, or any combination thereof.

7. The viral vector composition of claim 2, wherein a unique cell type and/or a unique cell state:
comprises a unique gene expression pattern;
comprises a unique anatomic location;
comprises anatomically locally unique gene expression;
comprises activation of one or more cellular activities of interest;
comprises activation of cellular activities associated with neural activity selected from the group comprising fear conditioning, memory formation, learning, sensory modalities, or any combination thereof,
is caused by hereditable, environmental, and/or idiopathic factors;
is caused by and/or associated with the expression of one or more endogenous proteins whose expression is regulated by the endogenous context-dependent promoter;
and/or is characterized by signaling of one or more endogenous signal transducer(s) regulated by the endogenous context-dependent promoter.

8. The viral vector composition of claim 2, wherein the unique cell state and/or unique cell type is characterized by:
one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment;
one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression; and/or
one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

9. The viral vector composition of claim 2, wherein the context-dependent promoter comprises a tissue-specific promoter and/or a lineage-specific promoter.

10. The viral vector composition of claim 9, wherein the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a hSynapsin promoter, a α-myosin heavy chain (a-MHC) promoter, or a cardiac Troponin T (cTnT) promoter, a neuronal activity-dependent promoter and/or a neuron-specific promoter, a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ 1.7 promoter, a Naᵥ 1.8 promoter, a Naᵥ 1.9 promoter, or an Advillin promoter.

11. The viral vector composition of claim 1, wherein the GVA genes and/or GVS genes are derived from Bacillus Megaterium, Anabaena flos-aquae, Serratia sp., Bukholderia thailandensis, B. megaterium, Frankia sp, Haloferax mediaterranei, Halobacterium sp, Halorubrum vacuolatum, Microcystis aeruginosa, Methanosarcina barkeri, Streptomyces coelicolor, and/or Psychromonas ingrahamii.

12. The viral vector composition of claim 1, wherein the one or more GV polynucleotides comprise:
two or more GVS genes derived from different prokaryotic species;
GVA genes and/or GVS genes from Bacillus Megaterium, Anabaena flos-aquae, Serratia sp., Bukholderia thailandensis, B. megaterium, Frankia sp, Haloferax mediaterranei, Halobacterium sp, Microchaete diplosiphon, Nostoc sp, Halorubrum vacuolatum, Microcystis aeruginosa, Methanosarcina barkeri, Streptomyces coelicolor, and/or Psychromonas ingrahamii;
gvpB, gvpN gvpF, gvpG, gvpL gvpS, gvpK, gvpJ, and/or gvpU from B. megaterium;
gvpA, gvpC, gvpN, gvpJ, gvpK, gvpF, gvpG, gvpV, and/or gvpW from Anabaena flos-aquae;
gvpR, gvpN, gvpF, gvpG, gvpL, gvpS, gvpK, gvpJ, gvpT and/or gvpU from B. megaterium and gvpA from Anabaena flos-aquae;
gvpA, and/or gvpC from Anabaena flos-aquae, and gvpN, gvpF, gvpG, gvpL, gvpS, gvpK, gvpJ, and/or gvpU from B. megaterium; and/or
gvpA, gvpC and/or gvpN from Anabaena flos-aquae, and gvpF, gvpG, gvpL, gvpS, gvpK, gvpJ, and/or gvpU from B. megaterium.

13. The viral vector composition of claim 1, the single viral vector comprises:
a first GV polynucleotide encoding GvpA, a second GV polynucleotide encoding GvpN, a third GV polynucleotide encoding GvpJ, a fourth GV polynucleotide encoding GvpK, a fifth GV polynucleotide encoding GvpF, a sixth GV polynucleotide encoding GvpG, a seventh GV polynucleotide encoding GvpW, and an eighth GV polynucleotide encoding GvpV,
and wherein each of the GV polynucleotides is operably connected to a tandem gene expression element.

14. The viral vector composition of claim 1, wherein the single viral vector is or comprises an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector derivatives thereof, or any combination thereof.

15. The viral vector composition of claim 1, wherein the single viral vector is:
a lentiviral vector selected from the group comprising human immunodeficiency virus 1 (HIV-1), human immunodeficiency virus 2 (HIV-2), visna-maedi virus (VMV) virus, caprine arthritis-encephalitis virus (CAEV), equine infectious anemia virus (EIAV), feline immunodeficiency virus (FIV), bovine immune deficiency virus (BIV), simian immunodeficiency virus (SIV), derivatives thereof, or any combination thereof, and/or
a recombinant lentiviral vector derived from a lentivirus pseudotyped with vesicular stomatitis virus (VSV), lymphocytic choriomeningitis virus (LCMV), Ross river virus (RRV), Ebola virus, Marburg virus, Mokala virus, Rabies virus, RD114, or variants therein.

16. The viral vector composition of claim 1,
wherein the single viral vector comprises one or more of a left (5′) retroviral LTR, a Psi (Ψ) packaging signal, a central polypurine tract/DNA flap (cPPT/FLAP), a retroviral export element, and a right (3′) retroviral LTR;
wherein the promoter of the 5′ LTR is replaced with a heterologous promoter;
wherein the 5′ LTR or 3′ LTR is a lentivirus LTR;
wherein the 3′ LTR comprises one or more modifications and/or deletions; and/or
wherein the 3′ LTR is a self-inactivating (SIN) LTR.

17. The viral vector composition of claim 2, wherein one or more GV polynucleotides and/or the transactivator polynucleotide comprise:
a 5′UTR and/or a 3′UTR;
a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof, and/or
a transcript stabilization element selected from the group comprising woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

18. The viral vector composition of claim 1, wherein the single viral vector is encapsidated in a viral particle.

19. A method of imaging gene expression within a subject, comprising:
administering to the subject an effective amount of the viral vector composition of claim 1; and
applying ultrasound (US) to the target site of the subject to obtain a US image of gene expression at the target site.

20. A method of detecting a unique cell type and/or unique cell state within a subject, comprising:
administering to the subject an effective amount of the viral vector composition of claim 1; and
applying ultrasound (US) to the target site of the subject, thereby detecting the unique cell type and/or unique cell state within said subject.
